(19) United States
(12) Patent Application Publication
(10) Pub. No.: US 2021/0052753 A1
(43) Pub. Date: Feb. 25, 2021

(54) METHODS AND APPARATUSES FOR PRESSURIZED PURIFICATION AND INFUSION OF PLANT MATTER AND CELLULOSE-BASED ORGANIC CELLULAR MATERIAL

(71) Applicant: PuroGen Laboratories, LLC, Winter Park, FL (US)

(72) Inventor: Alan J. Novotny, Orlando, FL (US)

(21) Appl. No.: 16/958,474

(22) PCT Filed: Dec. 31, 2018
(86) PCT No.: PCT/US2018/068107

§ 371 Date: Jun. 26, 2020

Related U.S. Application Data
(60) Provisional application No. 62/612,532, filed on Dec. 31, 2017.

(51) Int. Cl. A61L 2/00; A23B 7/005; A23B 7/148; A23L 3/015; A23L 3/3418; A23L 3/16; A23L 3/00; A24B 15/18; A24D 1/18

(52) U.S. Cl. CPC A61L 2/0094; A23B 7/0053; A23B 7/148; A23L 3/0155; A23L 3/3418; A61L 2202/21; A23L 3/001; A24B 15/183; A24D 1/18; A23V 2002/00; A23L 3/165

(57) ABSTRACT

A method includes heating a vacuum chamber to a first predetermined temperature, and providing an organic plant material within the vacuum chamber. A vaporizer is heated to a second predetermined temperature, and is in fluid communication with the vacuum chamber. The vacuum chamber is evacuated to a a first predetermined, sub-atmospheric pressure using a vacuum pump. A liquid reagent is injected into the vaporizer such that the liquid reagent transforms into a gaseous/aerosolized reagent. The gaseous/aerosolized reagent is introduced into the vacuum chamber. After waiting a predetermined duration, a sterilization of the organic plant material is achieved. The vacuum chamber is then vented to atmospheric pressure, and the vacuum chamber is again evacuated, to a second predetermined, sub-atmospheric pressure, to remove a reagent residue from the organic plant material, followed by a second venting of the vacuum chamber to atmospheric pressure.

### CROSS-REFERENCE TO RELATED APPLICATIONS

This application claims priority to and benefit of U.S. Provisional Patent Application No. 62/612,532, filed Dec. 31, 2017 and titled “Methods and Apparatuses for Pressurized Purification and Infusion of Plant Matter and Organic Cellular Materials,” the entire content of which is hereby expressly incorporated by reference for all purposes.

This application may contain material that is subject to copyright, mask work, and/or other intellectual property protection. The respective owners of such intellectual property have no objection to the facsimile reproduction of the disclosure by anyone as it appears in published Patent Office file/records, but otherwise reserve all rights.

### BACKGROUND

Sterilants are used in environments such as hospitals to render objects, such as medical instruments, free from potentially infectious living organisms. Sterilization is important for patient safety, particularly with regard to medical instrument and transplant tissue.

### SUMMARY

Embodiments of the present disclosure include, by way of non-limiting example, devices and methods for purifying cellulose-based organic cellular materials such as plant matter. In addition or alternatively, devices and methods described herein can be used for the infusion of materials, such as extracts, flavorants, essential oils, terpenes, etc., into cellulose-based organic cellular materials, including but not limited to plant matter.

According to some embodiments of disclosure, methods and systems for low temperature (e.g., about 18° C. to about 39° C.) reactive oxygen and/or oxygen-based sterilization is disclosed, providing environmentally green, cost-effective, energy efficient, rapid, and terminal sterilization solution for plants, botanicals, and the like. Methods, apparatuses, and systems of the disclosure provide gentle yet powerful decontamination of botanicals including, but not limited to the following: cannabis, spices, herbs, etc., including decontamination/removal of fungi, bacteria, and viruses. In doing so, the disclosure can facilitate the determination of a known shelf life for products, provide a “Certified Green” product to consumers, and/or reduce or limit liability by decontaminated products.

According to some embodiments, apparatuses of the disclosure can be installed at virtually any location. Some embodiments may be configured to utilize standard power, e.g., 220/240 VAC outlet, and not require additional facility additions or modifications. According to some embodiments, no outside venting is required, no building penetration is required, and/or no canisters of pressurized air, CO2, etc., are required, Some embodiments include an elegant device that, once attached to a power source, is ready to operate. Some embodiments of the disclosure include a method, comprising: heating a vacuum chamber to a first predetermined temperature; providing an organic plant material within the vacuum chamber, the organic material having a moisture content of from about 1% to about 40%; heating a vaporizer to a second predetermined temperature, the vaporizer in fluid communication with the vacuum chamber; performing, via a vacuum pump (e.g., a scroll pump or any other dry pump), a first evacuation of the vacuum chamber to a first predetermined, sub-atmospheric pressure; injecting a liquid reagent into the vaporizer such that the liquid reagent transforms into a gaseous/aerosolized reagent; introducing the gaseous/aerosolized reagent into the vacuum chamber; waiting a predetermined duration so as to achieve a sterilization of the organic plant material; performing a first venting of the vacuum chamber to atmospheric pressure; performing, via the vacuum pump, a second evacuation of the vacuum chamber to a second predetermined, sub-atmospheric pressure so as to remove a reagent residue from the organic plant material; and performing a second venting of the vacuum chamber to atmospheric pressure. In some implementations, the method further comprises performing, via the vacuum pump, a third evacuation of the vacuum chamber to a second predetermined, sub-atmospheric pressure so as to remove a reagent residue; and performing a third venting of the vacuum chamber to atmospheric pressure. In some embodiments, the sterilization comprises or results in at least a 50% bioburden reduction (reduction of harmful microbes such as mold, bacteria, fungus, etc.), at least a 60% bioburden reduction, at least a 70% bioburden reduction, at least a 80% bioburden reduction, at least a 90% bioburden reduction, at least a 95% bioburden reduction, at least a 97% bioburden reduction, at least a 98% bioburden reduction, at least a 99% bioburden reduction, at least a 99.5% bioburden reduction, and/or at least a 99.9% bioburden reduction. In some embodiments, a mold count is reduced to less than 50,000 colony-forming units (CFU), less than 25,000 CFU, less than 10,000 CFU, less than 5,000 CFU, less than 1,000 CFU, less than 500 CFU, less than 100 CFU, less than 50 CFU, and/or less than 10 CFU.

Some embodiments of the disclosure include a method, comprising: heating a vacuum chamber to a first predetermined temperature; providing an organic plant material within the vacuum chamber, the organic plant material having a moisture content of from about 0% to about 40%; heating a vaporizer to a second predetermined temperature, the vaporizer in fluid communication with the vacuum chamber; performing, via a vacuum pump, a first evacuation of the vacuum chamber to a first predetermined, sub-atmospheric pressure; injecting a liquid supplement into the vaporizer such that the liquid supplement transforms into a gaseous/aerosolized supplement; introducing the gaseous/aerosolized supplement into the vacuum chamber; and waiting a predetermined duration so as to achieve a infusion and/or saturation of the organic plant material with the supplement.

In some embodiments, the supplement includes at least one of: a cannabinoid oil, a terpenes, a terpinoid, a flavonoid, a cannaflavin, tetrahydrocannabinol (THC), and/or cannabidiol (CBD). In some embodiments, the organic plant material is cannabis plant material, including one or more of raw cannabis plant material, dried cannabis plant material, and/or cannabis flower.

Some embodiments of the disclosure include a method of reducing the bioburden of cannabis material and infusing said cannabis material with natural cannabis extracts to provide a sanitized organic cannabis product, the method comprising: obtaining organic cannabis material; processing the organic cannabis material such that the organic cannabis material has a moisture level between about 10% and about 16%; heating a pressure chamber to a first predetermined temperature via a first heater; inserting the organic cannabis material into the pressure chamber; heating a vaporizer via a second heater to a second predetermined temperature, the vaporizer in fluid communication with the pressure chamber; performing a first pressure change of the pressure chamber to a first predetermined pressure, the first predetermined pressure being a sub-atmospheric pressure; introducing a purifying, oxygen-based reagent into the pressure chamber via the heated vaporizer such that the purifying, oxygen-based reagent is in at least one of an aerosol, vapor, and/or gas form; processing the organic cannabis material in the pressure chamber with the purifying, oxygen-based reagent for at least one cycle having a predetermined duration, the processing reducing the bioburden of the organic cannabis material without irradiation; performing a first venting of the pressure chamber, the first venting raising the pressure of the pressure chamber to atmospheric pressure; performing at least one second pressure change of the pressure chamber to a second predetermined pressure to remove residue of the purifying, oxygen-based reagent from the organic cannabis material, the second predetermined pressure being a sub-atmospheric pressure; performing a second venting of the pressure chamber, the second venting raising the pressure of the pressure chamber to atmospheric pressure; heating the pressure chamber to a third predetermined temperature via the first heater; heating the vaporizer to a fourth predetermined temperature via the second heater; performing at least one third pressure change of the pressure chamber to a third predetermined pressure, the third predetermined pressure being a sub-atmospheric pressure; introducing a supplement into the pressure chamber via the vaporizer, the supplement being one or more natural cannabis extracts or components thereof; processing the organic cannabis material with the supplement in the pressure chamber for at least one infusion cycle having duration such that the organic cannabis material is infused with the one or more natural cannabis extracts or components thereof to produce a sanitized organic cannabis product; and outputting the sanitized organic cannabis product from the pressure chamber.

Some embodiments of the disclosure include at least one apparatus or system for performing one or more methods of the disclosure.

It should be appreciated that all combinations of the concepts discussed herein and detailed below (provided such concepts are not mutually inconsistent) are contemplated as being part of the inventive subject matter disclosed herein. In particular, all combinations of subject matter appearing in this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 illustrates an example system for purifying, hydrating, and/or infusing organic and biological material, according to some embodiments.

FIG. 2 illustrates an example apparatus for purifying, hydrating, and/or infusing organic and biological material, according to some embodiments.

FIG. 3 illustrates an example process flow, according to some embodiments.

FIG. 4 illustrates an example modular system for purifying, hydrating, and/or infusing organic and biological material, according to some embodiments.

FIGS. 5A-5B are photographs of an example apparatus for purifying, hydrating, and/or infusing organic and biological material, according to some embodiments.

FIG. 6 is a graph showing an example terpene analysis, according to some embodiments.

FIGS. 7A-7I show an example implementation of a system for purifying, hydrating, and/or infusing organic and biological material, including a user interface, menu and process screens, chamber, and reagent container, according to some embodiments.

### DETAILED DESCRIPTION

Following below are more detailed descriptions of various concepts related to, and embodiments of METHODS AND APPARATUSES FOR PRESSURIZED PURIFICATION AND INFUSION OF PLANT MATTER AND CELLULOSE-BASED ORGANIC CELLULAR MATERIALS. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

In some embodiments, a system and method for the enhanced purification of organic/cellular/biological materials, especially plant materials, is described. In other embodiments, a system and method for the infusion of one or more additives into purified organic/cellular/biological materials, especially purified plant materials, is described. In some embodiments, disclosed systems and methods can be used for both enhanced purification of, and infusion of one or more components/supplements/additives into, an organic/cellular/biological material, such as a plant material, and be conducted, for example, serially or substantially concurrently.

Effective sterilization of organisms is especially difficult in cannabis flower, for example since the sterilization and decontamination of cannabis can impact biomarkers such as THC, CBD, and terpenes such that they are undesirably reduced or no longer present in the sterilized/decontaminated cannabis flower. In some embodiments, a reactive oxygen (“rO”) system is configured to provide energy-efficient, effective, terminal decontamination of an organic/cellular/biological material, such as a plant material (e.g., cannabis), with minimal or no ecological footprint. The rO system can consistently purify, sterilize, disinfect, decontaminate, hydrate/re-hydrate, remediate mold, and/or reduce or eliminate microbes from, a batch of the material received within a vacuum chamber of the system, thereby producing a treated/finished product that is safe for human consumption (e.g., ingesting, smoking, or vaporizing). In some embodiments, one or more of the following microbes are reduced, inactivated, or substantially eliminated using the rO system: Geobacillus stearothemophilus, Bacillus atrophaeus (durable andospore, gram positive equivalent E. coli), Clostridium sporogenes, and Candida albicans (a fungal challenge organism). One or more supplemental materials can optionally be infused into the material using the rO system.

In some embodiments, an infusion process is preceded or accompanied by a hydration (or “re-hydration”) process or step. For example, a multi-step process can include a purification (or “sterilization”) step, a hydration step, and an infusion step, in any order, optionally with partial overlap in time and/or concurrent operation. In some embodiments, one or more walls of the vacuum/process chamber are heated during one or more of the multiple steps (purification, hydration, and infusion). Details of the purification process or step are set forth in the Purification section below, and details of the infusion process or step are set forth in the Infusion section below. The hydration process or step can include vaporizing a liquid or solvent, such as deionized (DI) water or reverse osmosis (RO) water, (e.g., under vacuum conditions set forth herein) such that the generated steam/vapor partially or fully hydrates the organic/cellular/biological material, such as a plant material, in-situ. This hydration process can be considered a re-hydration process, for example when the organic/cellular/biological material is a material that was previously dried. A “dried” material can be, for example, a material having a moisture content of about 4%, or between about 4% and about 11%, or between about 4% and about 7%, or between about 1% and about 5%, or between about 5% and about 10%. A material having a moisture level below 3% or 4% can be considered freeze dried, or nearly freeze-dried. The hydration process can be performed to achieve a desired/predetermined moisture level within the chamber and/or within the organic/cellular/biological material, and/or to manipulate moisture levels thereof in a desired direction (e.g., increase or decrease the moisture level(s)). In some embodiments, a material may be considered “fully hydrated” when it has reached a moisture level/content of up to 18%, for example about 12%, or about 15%, or between about 12% and about 18%, or between about 15% and about 18%, or between about 13% and about 17%, or between about 14% and about 16%.

As set forth herein, an infusion process can take a starting liquid material, such as a sterilant (e.g., about 35% hydrogen peroxide) or an essential oil, convert the starting liquid material into a vapor, and cause the vapor to penetrate a desired material (e.g., a plant material). Without wishing to be bound by theory, the penetration of the vapor into the material can be caused, for example, by a temperature gradient (e.g., where walls of a chamber in which a process occurs, the chamber walls may be at an elevated temperature (e.g., about 90° C.) with respect to the material itself (e.g., at about 70° C.). Depending on the implementation, the penetration of the vapor into the material can be complete (i.e., the material is fully penetrated by, or “saturated” with, the vapor), or can be partial.

In some embodiments, an infusion process or a multi-step process (at one or more stages/steps thereof) can include the introduction of one or more nutraceuticals into the chamber, such that is the one or more nutraceuticals are infused into, absorbed by, or otherwise incorporated into the material that is being processed. For example, L-Theanine can be infused into Indica to yield a finished product for use as a sleep aid or for anti-anxiety, and Theacrine can be infused into Sativa to yield a finished product for energy and focus. In some such implementations, the combination of the selection of the nutraceutical(s) and a selection of the material (e.g., a particular plant strain, terpene profile, concentration of a component of interest, etc.) can be used to produce a treated material (end product) having enhanced, synergistic properties (i.e., a “superflower”).

In some embodiments, an infusion process or a multi-step process can have an antimicrobial effect on the material being treated.

In some embodiments, an apparatus is configured to perform a process that includes low-temperature sterilization of a bulk material (such as cannabis flower) within a vacuum chamber using a reactive oxygen (vaporized H₂O₂, or “VH₂O₂”) sterilant. Generating the reactive oxygen can include hydrogen peroxide vaporization (HPV). The reactive oxygen can function as a broad-spectrum antimicrobial (e.g., achieving a 5-log microbial reduction), without causing condensation of any active ingredient onto the surface of the bulk material being treated. During processing, one or more of: temperature, humidity, pressure, process time, and reactive oxygen “dose” (e.g., partial pressure and/or flow rate) can be controlled (e.g., via a controller and according to a pre-programmed recipe) to ensure efficacy and/or repeatability. Byproducts of the process can be limited to water and oxygen, and as such, the process can be considered a completely organic sterilization process. In some embodiments, the reactive oxygen based process does not impact the THC, CBD and/or terpene composition/profile of the bulk material. In some implementations, one or more VH₂O₂ biological indicators, which contain a known population of Geobacillus stearothermophilus spores (e.g., ATCC 7953 or ATCC 12980), are used for process verification. For example, during a biodecontamination cycle of the processes set forth herein, the biolofical indicator can be inactivated by the reactive oxygen (hydrogen peroxide vapor). The inactivation can be verified using biological indicator medis, e.g., in 24-minute, 24-hour, or 7-day biodecontamination cycle results.

In some embodiments, a biodecontamination process (or phase of a multi-step process) includes a conditioning step, an exposure step, and optionally a post-conditioning step. During the conditioning step, a concentration of a reactive oxygen (vaporized hydrogen peroxide) sterilant is brought to a desired level (e.g., within a vaporizer or a vacuum chamber that, optionally, has been evacuated to a starting base vacuum/pressure level). The sterilant vapor can be introduced to (or generated within) the vacuum chamber by a vaporizer, which flash vaporized aqueous hydrogen peroxide solution and disperses it to airstream in a controlled manner. This flash vaporization can be used to increase a concentration of the vapor inside the enclosure as quickly as possible (e.g., to a level slightly below the point of saturation). The concentration can be gradually increased inside the vacuum chamber until a desired concentration and/or associated pressure has been achieved. The exposure step begins when the desired reactive oxygen vapor concentration has been achieved within the vacuum chamber. During the exposure step, the desired sterilant concentration (e.g., near-saturation) is maintained for a desired or pre-programmed period of time (e.g., according to a pre-programmed recipe and/or until a desired level of bioburden reduction has been achieved). An optional post-conditioning step, following the exposure step, can include aeration of the treated material by circulating air and reactive oxygen vapor throughout the vacuum chamber, to remove vapor from the load prior to ending the process cycle. During the post-conditioning, the vapor can be converted into water and oxygen molecules (e.g., using an integral catalytic converter system). Once the process has been completes, the chamber door can be opened to remove the finished product. The chamber door can be safely opened, for example, when sufficient time has elapsed and/or when the concentration of reactant has fallen to a sufficiently low level (e.g., as indicated by one or more measurement instruments).

Apparatuses of the present disclosure can include novel oxygen-based purification, including novel Moisture-Conducive Vaporized/Aerosolized Hydrogen Peroxide (MCVAHP) systems configured for processing plant materials (and/or the like) that contain moisture. The novel MCVAHP processes can be conducted without causing damage to the processed plant materials or to the MCVAHP apparatus. By contrast, existing sterilization methods, such as those typically used for sterilizing instruments in healthcare settings, cannot effectively process moisture-containing materials—failing to properly remove/neutralize contaminants and/or destroying/degrading the moisture-containing materials.

A variety of sterilization techniques are used in the medical industry, one of the most prevalent being irradiation. However, the irradiation process can damage certain important properties of moisture containing organic materials, such as plant materials, for example, by causing undesired chemical changes, including generating free radicals, and/or (e.g., in the case of case of cannabis) by altering or destroying a terpene profile thereof, which can result in a reduction in quality of the material. Hydrogen Peroxide Vaporization (HPV) is used in hospitals to sterilize instruments, such as batteries, that are moisture sensitive (i.e., instruments that a steam autoclave could damage). Such HPV systems are not typically equipped to handle moisture—typically including a dehumidifier and/or desiccant. If a high-moisture material were placed in such an HPV unit, the HPV unit would likely shut down with an error to prevent damage, and in any event, not be able to effectively process high-moisture material.

Moreover, it has been reported that mold, fungal, and/or bacterial contamination of cannabis or tobacco products can result in illness or death in those who consume it, for example individuals/patients who are immune-compromised. Medical cannabis is frequently used by chronically ill and/or immuno-compromised patients, and several recent studies have found retail cannabis, whether dried or raw, often has multiple bacterial and fungal pathogens that can cause serious infections, such as the fungi Cryptococcus, Mucor and Aspergillus, and the bacteria E. coli, Klebsiella pneumoniae and Acinetobacter baumannii (see, e.g., Thompson III, G. R., et al. “A microbiome assessment of medical marijuana.” Clin Microbiol Infect 23.4 (2017): 269-270.)

As such, users of cannabis, including medical and recreational cannabis, would benefit from reduction of microbial contamination, reducing the potential for opportunistic lung infections. While techniques such as ionizing radiation/irradiation, or heat sterilization/pasteurization could be used to for reducing contamination, they are often disfavored and include drawbacks. For example, such techniques typically require high energy, cause chemical changes, and/or cause the loss of important components such as low molecular weight compounds (e.g., terpenes, essential oils, flavors, etc.), when applied to plant materials such as cannabis or tobacco. In addition, many existing sterilization techniques are limited, only sterilizing the outside of plant materials. Since mold and mildew can originate and/or be present internally/within plant material, surface treatments are ineffective at addressing all possible contaminants.

Embodiments of the present disclosure utilize novel oxygen-based purification, including specialized Moisture-Conducive Vaporized/Aerosolized Hydrogen Peroxide (MCVAHP) technology. The disclosed MCVAHP systems and methods that are capable of handling high-moisture-content products (such as cannabis or tobacco), including at a moisture range from about 0% to 40%, 1% to 35%, 3% to 30%, 4% to 28%, 5% to 25%, 8% to 20%, or about 10% to 16% (w/w). While not wishing be bound by any particular theory, high-moisture materials as used herein can refer to plant material with more than 15%, more than 14%, more than 13%, more than 12%, more than 11%, more than 10%, more than 9%, more than 8%, more than 7%, more than 6%, more than 5%, more than 4%, more than 3%, more than 2%, or more than 1% moisture, either on a total weight basis, a wet weight basis, or otherwise, depending on the embodiment. The disclosed systems and methods are significantly more effective (i.e., 95%, 98%, or 99% more effective) at sterilizing and/or reducing the bioburden such plant materials than was previously possible, for example, capable of reducing a mold count from 600,000 CFU to less than about 100,000 CFU, less than about 75,000 CFU, less than about 50,000 CFU, less than about 40,000 CFU, less than about 30,000 CFU, less than about 20,000 CFU, less than about 15,000 CFU, less than about 10,000 CFU, less than about 9,000 CFU, less than about 8,000 CFU, less than about 7,000 CFU, less than about 6,000 CFU, less than about 5,000 CFU, less than about 4,000 CFU, less than about 3,000 CFU, less than about 2,000 CFU, less than about 1,000 CFU, less than about 900 CFU, less than about 800 CFU, less than about 700 CFU, less than about 600 CFU, less than about 500 CFU, less than about 400 CFU, less than about 300 CFU, less than about 200 CFU, less than about 100 CFU, less than about 90 CFU, less than about 80 CFU, less than about 70 CFU, less than about 60 CFU, less than about 50 CFU, less than about 40 CFU, less than about 30 CFU, less than about 20 CFU, less than about 10 CFU, less than about 9 CFU, less than about 8 CFU, less than about 7 CFU, less than about 6 CFU, less than about 5 CFU, less than about 4 CFU, less than about 3 CFU, less than about 2 CFU, less than about 1 CFU, or about 0 CFU.

Cannabis has long history of use for medicinal purposes, industrial purposes, and as a recreational drug. Industrial hemp products are made from cannabis plants selected to produce an abundance of fiber. Some strains have been bred to produce minimal levels of THC, the principal psychoactive constituent responsible for the psychoactivity associated with marijuana. Marijuana has historically consisted of the dried flowers of cannabis plants selectively bred to produce high levels of THC and other psychoactive cannabinoids. Various extracts including hashish and hash oil are also produced from the plant.

Cannabis plants produce a unique family of terpeno-phenolic compounds called cannabinoids. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. There are at least 483 identifiable chemical constituents known to exist in the cannabis plant (Rudolf Brenneisen, 2007, Chemistry and Analysis of Phytocannabinoids (cannabinoids produced by cannabis) and other Cannabis Constituents, In Marijuana and the Cannabinoids, El Sohly, ed.; incorporated herein by reference) and at least 85 different cannabinoids have been isolated from the plant. The two cannabinoids usually produced in greatest abundance are cannabidiol (CBD) and/or Δ9-tetrahydrocannabinol (THC). THC is psychoactive while CBD is not.

Cannabinoids are the most studied group of secondary metabolites in cannabis. Most exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. The biologically active forms for human consumption are the neutral forms. Decarboxylation is usually achieved by thorough drying of the plant material followed by heating it, often by either combustion, vaporization, or heating or baking in an oven. Unless otherwise noted, references to cannabinoids in a plant include both the acidic and decarboxylated versions (e.g., CBD and CBDA).

The cannabinoids in cannabis plants include, but are not limited to, Δ9-Tetrahydrocannabinol (Δ9-THC), Δ8-Tetrahydrocannabinol (Δ8-THC), Cannabichromene (CBC), Cannabicyclol (CBL), Cannabidiol (CBD), Cannabielsoin (CBE), Cannabigerol (CBG), Cannabinidiol (CBND), Cannabinol (CBN), Cannabitriol (CBT), and their propyl homologs, including, but are not limited to cannabidivarin (CBDV), Δ9-Tetrahydrocannabivarin (THCV), cannabichromevarin (CBCV), and cannabigerovarin (CBGV). Non-THC cannabinoids can be collectively referred to as “CBs”, wherein CBs can be one of THCV, CBDV, CBGV, CBCV, CBD, CBC, CBE, CBG, CBN, CBND, and CBT cannabinoids. Methods for administration of medical cannabis include, but are not limited, to vapor inhalation, smoking (e.g., dried buds), drinking, eating extracts or food products infused with extracts, and taking capsules.

As detailed herein, the novel MCVAHP methods, systems, and apparatuses can be utilized on organic materials, especially plant materials, such as cannabis flower material, to reduce, substantially eliminate, essentially eliminate, or eliminate harmful microbes and/or the risk therefrom for legal users, while providing supplements to said organic materials.

### Purification:

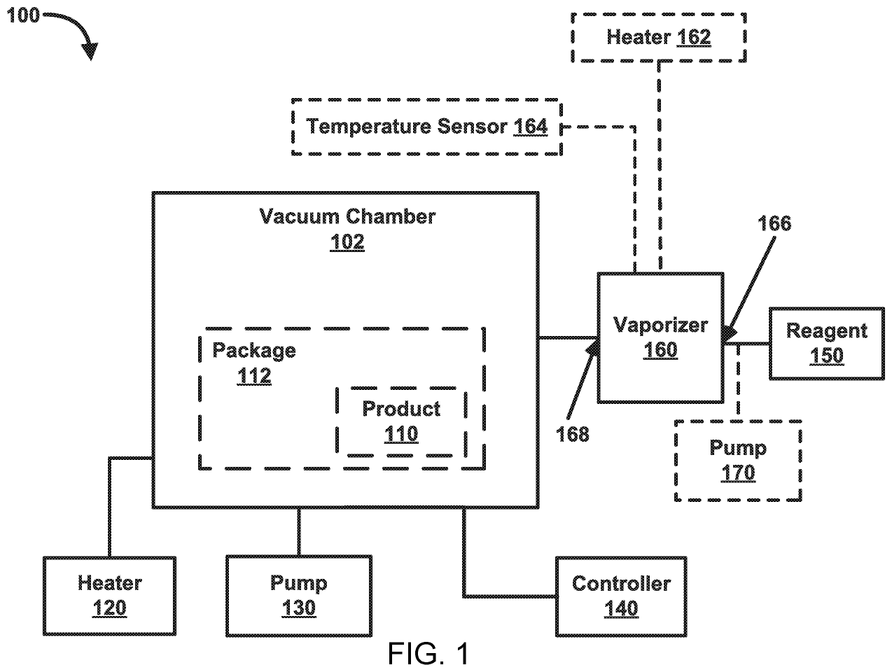

FIG. 1 illustrates an example system 100 for purifying, hydrating, and/or infusing an organic plant material, according to some embodiments. In some embodiments, a plant product 110 (i.e., one or more items) to be purified (also referred to herein as “sterilized”) is placed into a package 112. The package 112 can comprise medical-grade materials including, for example, a semi-permeable material (e.g., Tyvek) on one side and a clear plastic cover on an opposing side. While some embodiments use a package, other embodiments can use a different container or product holder. Once the plant product 110 has been properly prepared/packaged, it can be placed into the system's vacuum chamber 102 (for example, having a size to accommodate one or more packages, up to 200 packages, each package having a mass when filled with plant product material of 0.5 grams to 5 kg). The chamber 102 can have any capacity suitable for the processing of a plant material (e.g., up to or exceeding about 5 pounds of plant material). In some such embodiments, the vacuum chamber 102 can be pre-heated (e.g., by a heater 120) to a temperature in a range from about 20° C.-55° C. The temperature can selected based on the material, and the system configured accordingly. After placing the product 110 in the vacuum chamber 102, the door to the vacuum chamber 102 is closed and sealed. The heated chamber 102 provides an environment in which a subsequently-introduced reagent 150 can become evenly dispersed throughout the chamber 102 and/or the product 110, in some implementations, via the package 112.

Once the product 110 (such as in package 112) has been loaded into the vacuum chamber 102 and the vacuum chamber 102 has been sealed, a processing procedure (e.g., via a software program implemented on one or more processors of the system) is initiated, for example using a controller 140. Controller 140 can include a compute device having memory, data stores, one or more processors, interfaces, inputs/outputs, etc., for example, keyboard, touchscreen, displays, graphical user interface(s), a Human Machine Interface (HMI) screen. The system 100 can be controlled via one or more Programmable Logic Controllers (PLC's), e.g., utilizing Ladder Logic. Within the software, one or more variables of the system's operation can be controlled.

In one embodiment, a “purification” process is performed over the course of a cycle having a duration, for example, of about 1 minute to about 6 hours, including about 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 60 minutes, 70 minutes, 80 minutes, 90 minutes, 120 minutes, 150 minutes, 180 minutes, 240 minutes, 300 minutes, or about 360 minutes, etc., in some implementations, from about 16 minutes to about 42 minutes.

An exemplary process logic for a purification process according to the disclosure can begin as follows: Heat the vacuum chamber 102 (e.g., to temperature from about 15° C. to about 70° C., including about 20° C., about 25° C., about 30° C., about 35° C., about 40° C., about 45° C., about 50° C., about 55° C., about 60° C., or about 65° C.); Heat the vaporizer/aerosolizer 160 (e.g., from about 15° C. to about 250° C., depending on the reagent to be vaporized/aerosolized), e.g., using heater 162, and/or monitoring a temperature of the vaporizer 160 using a temperature sensor 164; Load the reagent/reagents (“reagent”) 150 (e.g., one or more oxygen-based reagents, such as H2O2) at the appropriate concentration (e.g., for H2O2 at 3% to 35% concentration) into a reagent receptacle; Prime the reagent 150; Evacuate the vacuum chamber 102 (i.e., “activate” vacuum, to a base pressure, depending on the implementation, from about 1 Torr to about 750 Torr); and Inject the reagent 150 into the vaporizer/aerosolizer 160 (e.g., from about 1 cc to about 25 cc of reagent). In some embodiments, reagent can be pumped via a liquid reagent input port 166 of the vaporizer/aerosolizer 160 using a reagent pump 170 or actuator. According to some embodiments, during an injection process, the reagent 150 is transformed from a liquid to a gas/vapor/aerosol; and the reagent gas/vapor/aerosol can be introduced into the vacuum chamber 102 via a gaseous reagent output port 168 of the vaporizer/aerosolizer 160. It should be understood that variables of the system's operation can be controlled throughout the process, including manually and/or programmatically, and may or may not be guided/controlled by feedback from one or more sensors, monitors, etc.

According to some embodiments, the product will then dwell—i.e., a cycle or series of cycles provide sufficient time for the gaseous/vaporized/aerosolized reagent to diffuse into the product 110 and/or diffuse through the package 112 and penetrate into the product 110.

In some embodiments, a purification cycle or cycles can have a duration of 30 seconds, 45 seconds, 60 seconds, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 30 minutes, 45 minutes, 60 minutes, 90 minutes, 120 minutes, 150 minutes, 180 minutes, 240 minutes, 300 minutes, 360 minutes, 420 minutes, 480 minutes, 540 minutes, or any integers there between. In some embodiments, the methods and systems of the disclosure can be configured such that the total purification time of all purification cycles is about 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 30 minutes, 45 minutes, 60 minutes, 90 minutes, 120 minutes, 150 minutes, 180 minutes, 240 minutes, 300 minutes, 360 minutes, 420 minutes, 480 minutes, 540 minutes, or any integers there between.

In some embodiments, a purification process or step can, alternatively or in addition to the foregoing, include exposing the material to ultraviolet (UV) radiation, for example to sterilize or de-contaminate at least a surface of the material.

The disclosed systems and methods can include one or more pressure variations (controlled by pumps, valves, etc.) to which the product is exposed to during processing, based on determined pressure parameters. The pressure(s) can be target pressures and/or set point pressures, and it is to be understood that the pressures of the disclosure are typically following a curve or line as the pressure changes from an initial pressure (e.g., atmospheric or external pressure) to or towards the pressure(s) based on the determined pressure parameters. For example, some embodiments can vary the pressure in the vacuum chamber 102, and the gaseous/vaporized/aerosolized state of the reagent can be altered by varying the pressure (e.g., between about 1 Torr and about 750 Torr and/or any integers there between). In some embodiments of the disclosure, one or more cycles are performed (including cycles of varying time/duration and pressure(s)) to saturate the product 110 with the reagent 150.

In some embodiments, the vacuum chamber 102 can be vented, generally to atmospheric pressure (i.e., about 760 Torr) and exhaust the reagent 150 out of the vacuum chamber 102 (either to atmosphere and/or to a recovery/reclamation device). In some embodiments, a “cleaning” step or cycle is performed, involving one or more (e.g., two) additional vacuum cleaning stages, to eliminate any residual/remaining reagent(s) from the product 110.

Infusion:

In some embodiments, a product (e.g., cannabis) in which one or more components/supplements/additives is to be infused is placed into a package, or remains in the package used in the purification process above. In some embodiments, similar to the purification process described above, and still referring to FIG. 1, the package 112 can comprise medical-grade materials including, for example, a semi-permeable material (e.g., Tyvek) on one side and a clear plastic cover on an opposing side. Once the product 110 has been properly packaged in the package 112, it can be placed into the vacuum chamber 102 (for example, having a size of 500 grams per package totaling between 5-25 lbs per cycle/run) and/or remain in the vacuum chamber 102 following purification. In some such embodiments, the vacuum chamber 102 is pre-heated to a temperature in a range from about 20° C.-55° C. If open, the door to the vacuum chamber 102 is closed and sealed. The heated chamber 102 provides an environment in which the subsequently-introduced one or more components/supplements/additives 151, such as essential oils and/or extracts, can become evenly dispersed throughout the chamber 102.

Once the product 110 has been loaded into the vacuum chamber 102 and the vacuum chamber 102 has been sealed, software is initiated as discussed above, for example using a controller 140 and/or one or more input/output devices/interfaces. The system 100 can, in some embodiments, be controlled via a PLC, e.g., utilizing Ladder Logic. Within control software, one or more variables of the system's operation can be controlled.

In one embodiment, an “infusion” process is performed over the course of a cycle having a duration, for example, of about 8 minutes to about 25 minutes. An exemplary process logic for an infusion process is as follows:
Heat the vacuum chamber 102 (e.g., as discussed above, and depending on the component(s), supplement(s), and/or additive(s)—generally “supplement”);
Heat the vaporizer 160 (e.g., again, as discussed above and depending on the supplement to be infused), e.g., using heater 162 and/or monitoring a temperature of the vaporizer/aerosolizer 160 using a temperature sensor 164;
Load the supplement 151;
Prime the supplement 151;
Evacuate the vacuum chamber 102 (i.e., “activate” vacuum, e.g., to a base pressure of between about 3 Torr to about 750 Torr);
Inject the supplement 151 (e.g., into the vaporizer 160);
Dwell: Wait a sufficient time for the vaporized/aerosolized/gaseous supplement 151 to diffuse through the package 112 and penetrate into the product 110.

As discussed above with the purification process, in some embodiments, the pressure and/or temperature can be varied, over one or more cycles/sub-cycles, such that the product is properly and adequately infused with supplement. In some embodiments, the infusion process can include a cycle at a pressure greater than atmospheric pressure. In some embodiments, unlike the purification process, the infusion process does not conclude with (or include at all) a “cleaning” cycle or step. During the infusion process “priming”, a liquid supplement can be primed from a bottle into the vaporizer. In some embodiments, the supplement can be recovered from a recovery/reclamation device (i.e., replacing one or more components of the product that was lost and captured during purification. In some embodiments, the vaporizer is constructed from one or more metals or metal allows, such as 6061 aluminum. The liquid supplement can include, but is not limited to: one or more essential oils or a blend thereof, an extract or synthetic equivalent of a natural component of the product, such as, for cannabis, one or more cannabinoid oils, terpenes, terpinoids, flavonoids, cannaflavins, tetrahydrocannabinol (THC), cannabidiol (CBD), which may be isolated, in combination, and/or in solution with a base or carrier, H2O, and/or the like.

As shown in FIG. 1, depending upon the embodiment, the vaporizer 160 includes ports for one or more of the following:
a heater 162 (e.g., a 220V, cartridge-style electric heater);
a temperature sensor/gauge 164;
a liquid input port 166; or
a gas output port 168.

The liquid reagent 150 and/or supplement 151 is injected into the heated vaporizer 160 chamber. The vaporizer 160 is connected to (i.e., is in fluid communication with) the vacuum chamber 102, and a vacuum pressure of the vaporizer 160 is identical to or substantially the same as the pressure of the vacuum chamber (i.e., whatever the pressure set-point of the vacuum chamber, the same or similar pressure is present inside the vaporizer). In some embodiments, the presence of a “vacuum” (i.e., a pressure that is below atmospheric pressure) inside the vaporizer 160 chamber facilitates the vaporization of liquids at lower temperatures than would be sufficient if the process were performed at atmospheric pressure. The pressure and/or temperature can be adjusted to optimize the process based on the type of liquid reagent 150 and/or supplement 151 being used.

When the liquid reagent 150 and/or supplement 151 is injected into the vaporizer 160 chamber, it can be instantly, substantially instantly, or quickly vaporized/aerosolized into a gaseous state/aerosol and drawn/pulled into the vacuum chamber 102, and migrates toward one or more lower-temperature surfaces within the vacuum chamber 102, and in some embodiments is ultimately attracted to the product 110, as the product can be the coolest location within the vacuum chamber 102. In some embodiments, the product can be chilled prior to processing.

### Example I

An example purification process was performed, using a vaporizer temperature of about 110° C. (i.e., such that the reactant gas migrates to one or more lower-temperature regions within the vacuum chamber), a vacuum chamber temperature of about 40° C. [gas moves again to lower temp area], and a product temperature (inside the product package) of about 20° C. to about 30° C.).

### (1) Diffusion

During the purification process, the reagent gas was diffused into the product at different pressures, since changes in pressure affect the state of the gas in the process (i.e., the lower the vacuum, the dryer the gas; the higher the vacuum, the greater the moisture content of the gas). The reagent gas was driven/pushed toward the center of the product under high vacuum (e.g., a first pressure value of about 3 Torr to about 100 Torr) and retained there for a first predetermined exposure duration. After the first predetermined exposure duration has elapsed, the vacuum chamber pressure is increased to a first increased pressure value (e.g., to about 50 Torr to about 200 Torr) and held at the first increased value for a second predetermined exposure duration. After the second exposure duration has elapsed, the vacuum chamber pressure is again raised to a second increased pressure value (e.g., to about 250 Torr to about 600 Torr) and held at the second increased value for a third predetermined exposure duration. The first predetermined exposure duration, the second predetermined exposure duration, and the third predetermined exposure duration correspond to three distinct stages of diffusion during which purification (and/or, in some embodiments, infusion) occurs.

### (2) Cleaning

The final stage of the purification process included venting the residual/remaining gas out of the vacuum chamber by venting the vacuum chamber to atmospheric pressure (about 760 Torr). Two substantially identical vacuum processes were then performed, in which the vacuum chamber was evacuated to a pressure of about 3-700 Torr (i.e., a “holding pressure”) and held at that pressure value for a given period (here, between about 5 second and about 30 seconds, though it can be different or the same for other embodiments). The vacuum chamber was then vented back to atmospheric pressure. As noted above, these final two steps are used in the purification process to remove any remaining reagent, but in most embodiments are not used in infusion processes of the present disclosure, since the intention with infusion is to retain the reagents within the product.

The present disclosure contemplates that, in some instances, systems, apparatuses, and/or methods described above can be combined such that a product received with a vacuum chamber receives both purification and infusion treatments, for example either sequentially/serially or substantially concurrently.

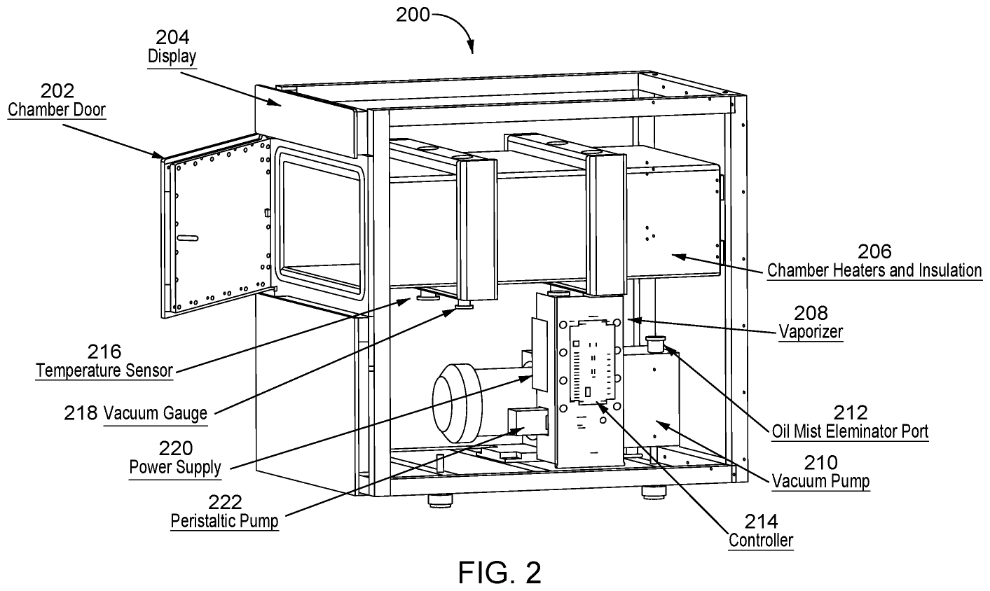

FIG. 2 illustrates an example apparatus for purifying, hydrating, and/or infusing organic and biological material, according to some embodiments. As shown in FIG. 2, the apparatus 200 includes a housing with a chamber door 202 and a display 204 (e.g., including a graphical user interface (GUI)) affixed thereto. A vacuum chamber, with chamber heaters and insulation 206 is disposed within the housing and accessible via the chamber door 202. A temperature sensor 216 and a vacuum gauge 218 are disposed on brackets holding the chamber 206 in place, and are at least one of in physical contact with or in fluid communication with the chamber 206. Also included within the housing are a vacuum pump 210, in fluid communication with the chamber 206 and optionally including an oil mist eliminator port 212; a controller 214; a peristaltic pump 222; a vaporizer 208; and a power supply 220 (for supplying power to one or more of: the vacuum pump 210, the controller 214, the peristaltic pump 222, the chamber heaters 206, the vaporizer 208, the vacuum gauge 218, the temperature sensor 216, and the display 204). Although some of the foregoing components are shown and described as being co-located within a common housing, it is to be understood that other configurations, including configurations in which some (i.e., any subset thereof) or all of the components are positioned without or outside a housing, are also contemplated.

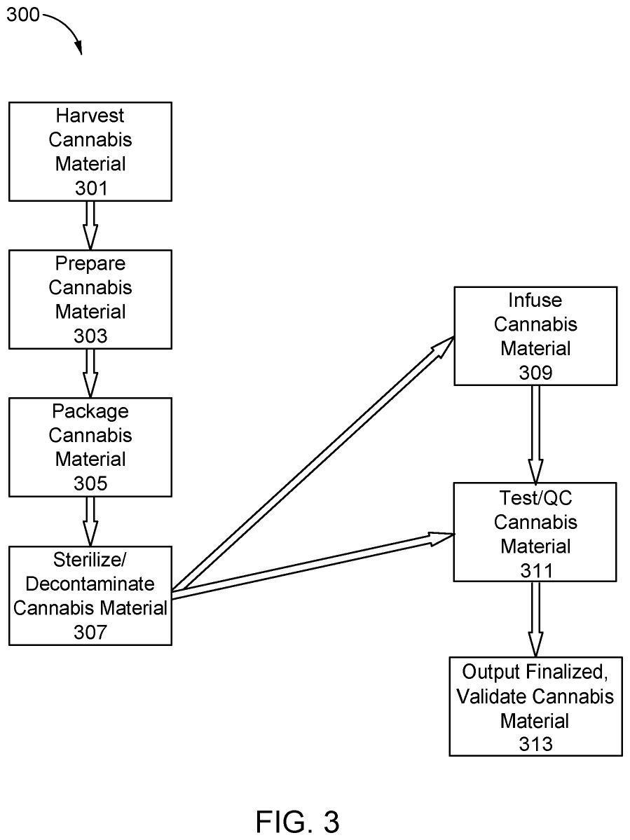

FIG. 3 illustrates an example process flow, according to some embodiments. As shown in FIG. 3, the process 300 includes harvesting cannabis material at 301, preparing the cannabis material at 303, packaging the cannabis material 205, and sterilizing/de-contaminating the cannabis material at 307 (optionally with heated chamber walls during the sterilization process). After sterilizing/de-contaminating the cannabis material at 307, the process 300 can either further include infusing cannabis material at 309, testing (i.e., performing quality control (QC)) the cannabis material at 311, and outputting the finalized, treated, and validated cannabis product (finished product) at 313, or the process 300 can proceed directly to testing the cannabis material at 311 and outputting the finished product at 313 without the infusion step 309.

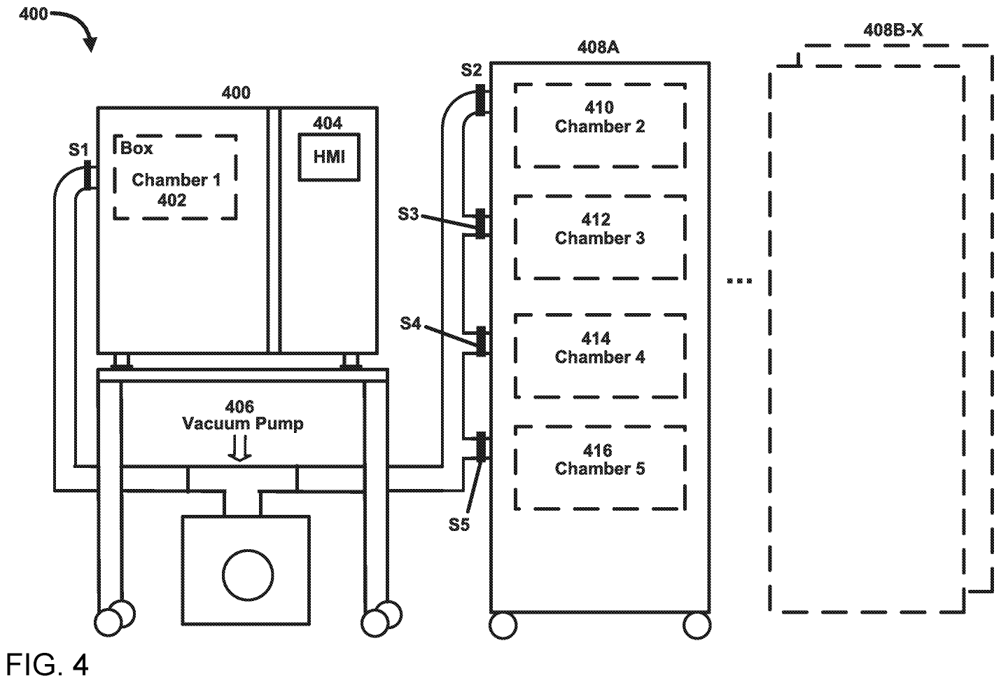

FIG. 4 illustrates an example modular system for purifying, hydrating and/or infusing organic and biological material, according to some embodiments. As shown in FIG. 4, the modular system 400 includes an apparatus 400 (which may be similar to the apparatus 200 of FIG. 2) having a vacuum chamber 402 (“Chamber 1”) and a human-machine interface (HMI) 404 (e.g., a GUI or other input/display component). The apparatus 400 (a “table top control unit”) is mounted on a table, beneath which a vacuum pump 406 is disposed. The vacuum pump 406 is in fluid communication, via tubing/piping and vacuum connectors, with the chamber 402 and configured, during operation, to pull a vacuum on the chamber 402. The HMI 404 can be communicatively coupled to a processor and a memory storing processor-executable instructions to perform process recipes within the apparatus 400 (and, more specifically, within the chamber 402). The processor and/or memory can be disposed within the housing of the apparatus 400, attached directly thereto (i.e., hardwired), or communicably accessible via a wired or wireless network connection. The modular system 400 further includes at least one further vacuum chamber 410, 412, 414, 416 (chambers 2-5) also in fluid communication with the vacuum pump 406, such that the vacuum pump 406 can evacuate (pull vacuum on) the at least one further vacuum chamber (410, 412, 414, 416). As shown in FIG. 4, each of the vacuum chambers 402, 410, 412, 414 and 416 is coupled to the vacuum pump 406 via a pipe or tube via an associated valve (e.g., solenoid valves) at S1-S5, respectively. The HMI 404 can also be used to control process recipes occurring in the at least one further vacuum chamber (410, 412, 414, 416), in addition to those taking place in vacuum chamber 402. For example, as part of a given process recipe, the HMI 404 may control one or more of the valves S1-S5, the vacuum pump 406, and/or one or more additional components (not shown) that may include, but are not limited to, a heater, a vaporizer, a gas inlet (e.g., for oxygen and/or nitrogen), a mass flow controller, a liquid inlet (e.g., for DI or RO water), a vacuum gauge or sensor, a pressure gauge, a temperature sensor, etc. The at least one further vacuum chamber (410, 412, 414, 416) can be housed within a common enclosure (e.g., a cart, rack, or “floor stand”) 408A, and one or more additional enclosures 408B-X, with associated additional one or more vacuum pumps, are optionally included, such that the system 400 is expandable/scalable. Modular systems such as system 400 facilitate increased processing throughput of materials, since multiple “batches” of the material, or multiple different types of material, can be concurrently processed within vacuum chambers 402 and 410-416.

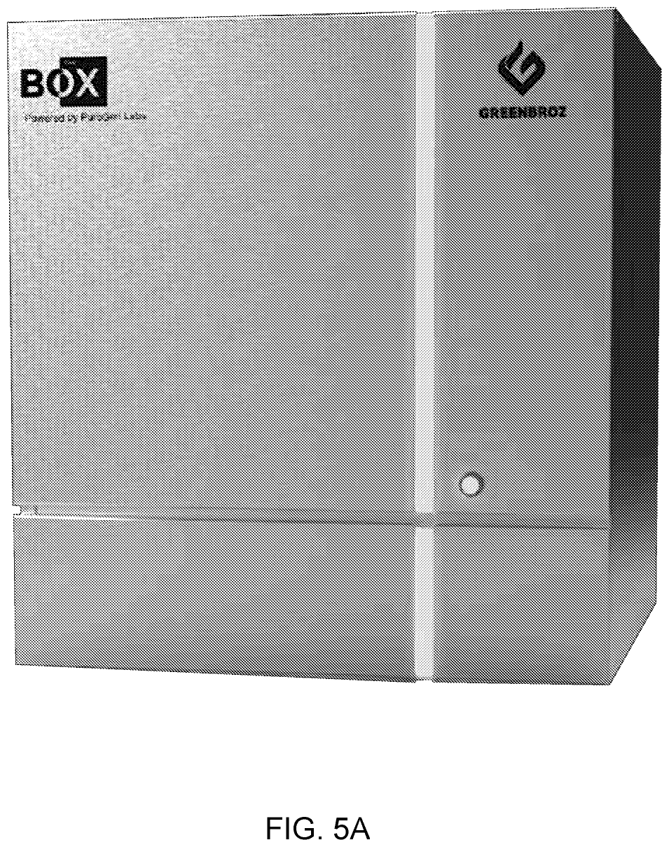

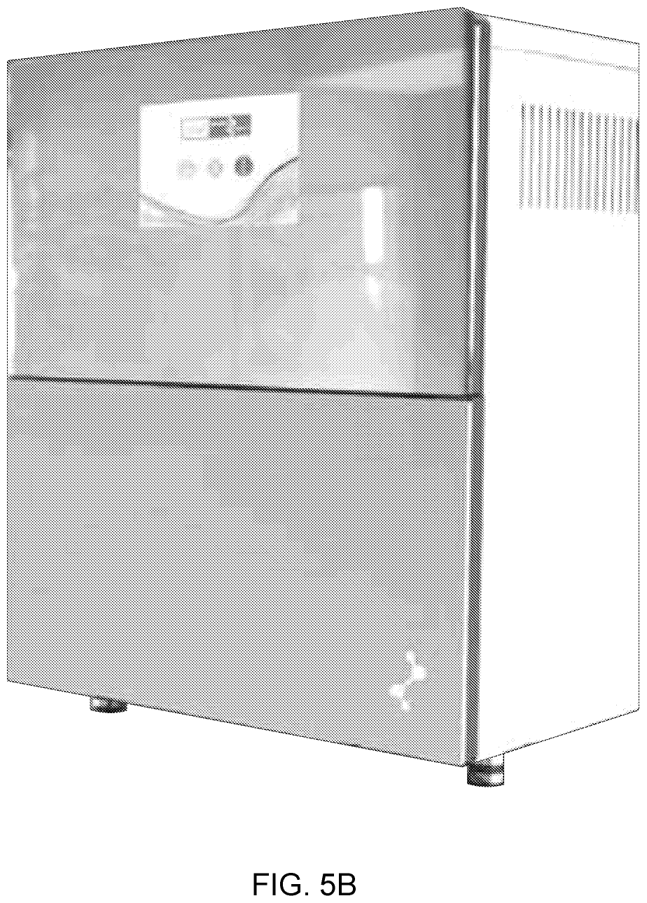

FIGS. 5A-5B are photographs of an example apparatus for purifying, hydrating and/or infusing organic and biological material, according to some embodiments. In some embodiments, an apparatus configured to perform purifying, hydrating and/or infusing processes has one or more of the following properties: a recipe run time of about 17 minutes or less, a throughput of up to about 5 pounds of material per chamber per run, a size of about 32″ tall by about 32″ wide by about 36″ deep, a weight of about 154 pounds, an ability to eliminate up to 600,000 CFU of pathogens during a purification run, and a capability (e.g., a processor-implemented capability) to track one or more of a number of runs, process run results, user information, equipment status, etc. (e.g., with a smart phone or other portable compute device in operable communication with the apparatus or a controller thereof).

In some embodiments, an apparatus configured to perform purifying, hydrating and/or infusing processes has the characteristics shown in Table 1 below:

TABLE 1
Example Apparatus Characteristics

| Type of Technology | Reactive Oxygen |
|---|---|
| Packaged Sterilization Option | Yes |
| Flower Infusion with Terpenes, | Yes |
| Essential Oil, & Nutraceuticals | |
| Low Temperature Sterilization | Yes |
| Process Time | 17 minutes |
| Purification to Center of Flower | Yes |
| Maximum CFU Ability | 600,000 |
| In-Process Proof of Sterilization | Yes |
| (BI) | |
| Purification of Visible Powder | Yes |
| Mildew | |
| Batch Size | 5-7 pounds |
| Nitrogen Preservation | Yes |

### Example Validation Data

Three batches of cannabis (Dream Queen strain) and five batches of cannabis (Kings Kush strain) were processed using an apparatus and method of the present disclosure, and the final, sterilized product (the processed cannabis) was analyzed for cannabinoid preservation. The results are shown in Table 2 below.

TABLE 2
Example Cannabinoid Preservation Data

| | | | B | | Caryo- | Limo- | |
|---|---|---|---|---|---|---|---|
| STRAIN | | THCa | Myrcene | Ocimene | philene | nene | Pinene |
| Dream | Before | 22.9 | 22.7 | 5.9 | 3 | 1.5 | 1.3 |
| Queen | After | 24.8 | 21.9 | 4.5 | 2.6 | 1.4 | 1.2 |
| Dream | Before | 26.3 | 22.4 | 5.8 | 3 | 1.5 | 1.4 |
| Queen | After | 21.6 | 21.1 | 4.5 | 2.9 | 1.4 | 1.3 |
| Dream | Before | 23 | 21 | 5.3 | 2.8 | 1.4 | 1.2 |
| Queen | After | 23.2 | 20.8 | 4.7 | 2.7 | 1.4 | 1.2 |
| King's | Before | 21.8 | 26.7 | 4.7 | 2.7 | 1.3 | 0.7 |
| Kush | After | 25.3 | 25.8 | 4 | 2.5 | 1.1 | 0.7 |
| King's | Before | 22.3 | 22.6 | 5.7 | 2.8 | 1.4 | 1.3 |
| Kush | After | 22.2 | 22.9 | 5.5 | 2.7 | 1.5 | 1.2 |
| King's | Before | 24.4 | 23.6 | 5.5 | 2.1 | 1.2 | 1.4 |
| Kush | After | 24.1 | 23.9 | 5.5 | 2.2 | 1.1 | 1.2 |
| King's | Before | 24.2 | 18.1 | 4.6 | 2.5 | 1.4 | 1.2 |
| Kush | After | 23.2 | 18.0 | 4.8 | 2.1 | 1.2 | 1.1 |
| King's | Before | 22.9 | 23.6 | 5.5 | 2.6 | 1.5 | 1.3 |
| Kush | After | 22.2 | 22.9 | 5.6 | 2.2 | 1.3 | 1.3 |

Six batches of cannabis (Rug Burn strain) were processed using an apparatus and method of the present disclosure, and the final, sterilized product (the processed cannabis) was analyzed for potency preservation. The results are shown in Table 3 below.

TABLE 3
Example Potency Preservation Data

| BATCH | | THCa | THC | CBN | CBD | CBDa |
|---|---|---|---|---|---|---|
| 1 | Pre-Sterilization | 22.75% | 1.62% | 0.17% | 0.12% | 1.40% |
| | Post-Sterilization | 22.05% | 1.91% | 0.13% | 0.10% | 1.42% |
| 2 | Pre-Sterilization | 22.99% | 1.82% | 0.17% | 0.10% | 1.50% |
| | Post-Sterilization | 22.05% | 1.83% | 0.16% | 0.10% | 1.43% |
| 3 | Pre-Sterilization | 23.85% | 1.69% | 0.20% | 0.11% | 1.47% |
| | Post-Sterilization | 23.05% | 1.70% | 0.19% | 0.10% | 1.43% |
| 4 | Pre-Sterilization | 23.78% | 1.70% | 0.17% | 0.12% | 1.52% |
| | Post-Sterilization | 23.80% | 1.69% | 0.16% | 0.11% | 1.53% |
| 5 | Pre-Sterilization | 22.25% | 1.81% | 0.16% | 0.12% | 1.49% |
| | Post-Sterilization | 22.05% | 1.81% | 0.15% | 0.11% | 1.43% |
| 6 | Pre-Sterilization | 22.05% | 1.62% | 0.16% | 0.11% | 1.44% |
| | Post-Sterilization | 22.06% | 1.89% | 0.16% | 0.10% | 1.43% |

Fifteen batches/sample of dried cannabis flower were processed using an apparatus and method of the present disclosure, and the final, sterilized product (the processed cannabis) was analyzed for moisture content (using an Ohaus MB23), terpene preservation (using a 7820A/5977B gas chromatograph-mass spectrometry (GC-MS)), and microbial load. Of all samples analyzed, none fluctuated more than +/−1% in moisture content. In other words, no significant change in moisture content was observed between the pre-sterilization cannabis flower and the post-sterilization cannabis flower, for the same set of process/program parameters (recipe).

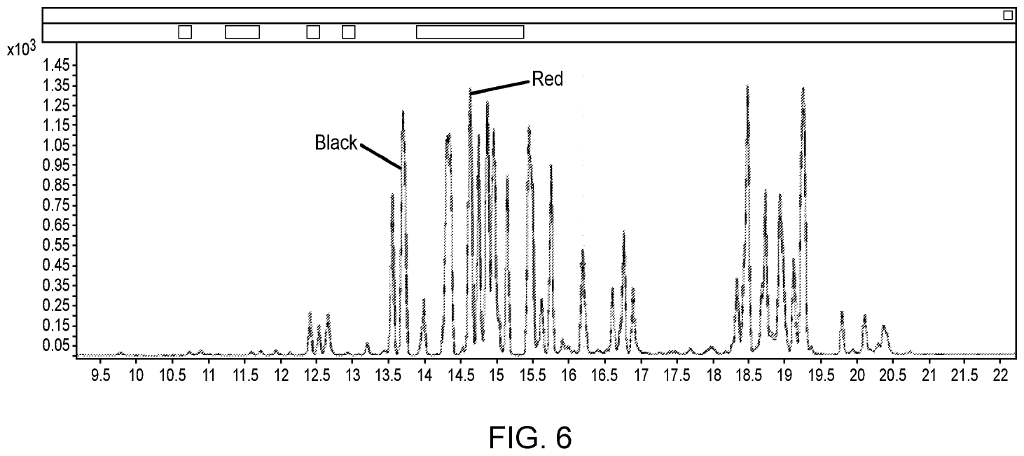

FIG. 6 shows an overlay of two chromatograms analyzing terpenes of the samples, with each peak corresponding to an individual terpene in the cannabis plant. The chromatogram labelled “red” is associated with the pre-sterilized cannabis flower, and the chromatogram labelled “black” is associated with the post-sterilized cannabis flower. As can be observed in FIG. 6, there is no significant change in terpene profile between the pre- and post-sterilization cannabis flower.

Six batches of cannabis (Sour Diesel strain) and seven batches of cannabis (OG Kush strain) were processed using an apparatus and method of the present disclosure, and the final, sterilized product (the processed cannabis) was analyzed for microbial load. The microbial load testing was performed using a modified USP,61> and <62> method for determination of total yeast and molds (Saboraud dextrose agar), total aerobic bacteria (tryptic soy agar), Salmonella (xylose lysine deoxycholate agar), E. coli (MacConkey agar), and S. aureus (Mannitol salt agar). The results are shown in Table 4 below.

TABLE 4
Example Microbial Testing Data

| | | | Aerobic | Aerobic |
|---|---|---|---|---|
| | Mold, | Mold, | Bacteria, | Bacteria, |
| | Pre- | Post- | Pre- | Post- |
| STRAIN | Purification | Purification | Purification | Purification |
| Sour Diesel | 100,000 | 3,000 | 180,000 | 0 |
| Sour Diesel | 27,500 | 0 | 12,000 | 0 |
| Sour Diesel | 47,000 | 8,070 | 110,000 | 100 |
| Sour Diesel | 130,000 | 1,000 | 160,000 | 45 |
| Sour Diesel | 37,000 | 0 | 0 | 0 |
| Sour Diesel | 25,800 | 0 | 0 | 0 |
| OG Kush | 33,000 | 4,200 | 80,000 | 0 |
| OG Kush | 110,000 | 3,330 | 4,000 | 0 |
| OG Kush | 260,000 | 1,040 | 72,000 | 0 |
| OG Kush | 165,000 | 0 | 180,000 | 0 |
| OG Kush | 84,000 | 3,010 | 180,000 | 0 |
| OG Kush | 750,000 | 750 | 120,000 | 500 |
| OG Kush | 172,000 | 3,700 | 180,000 | 0 |

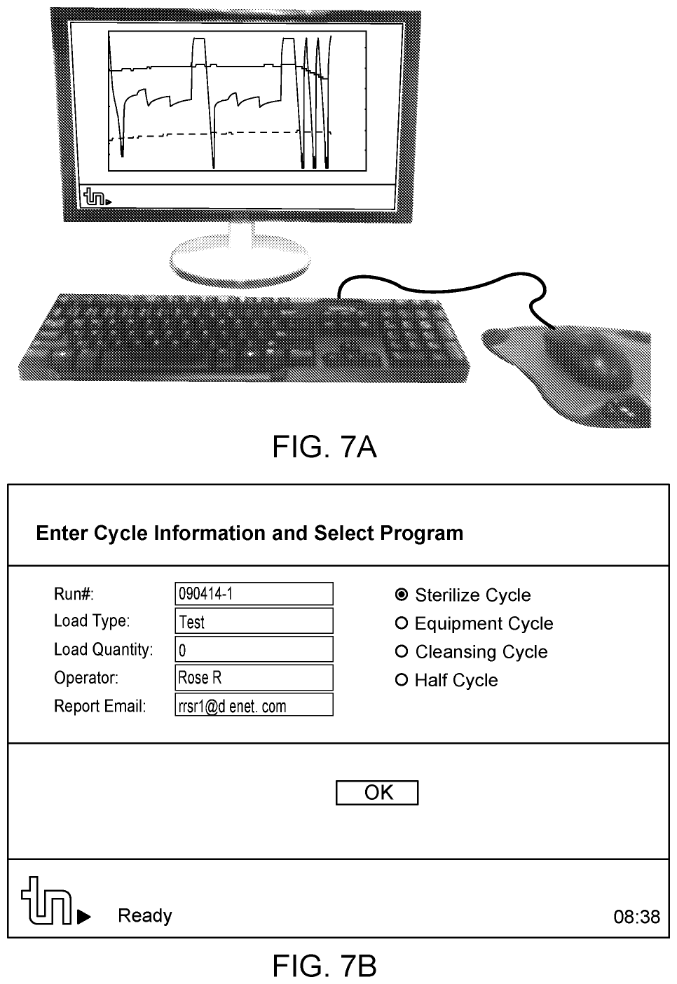

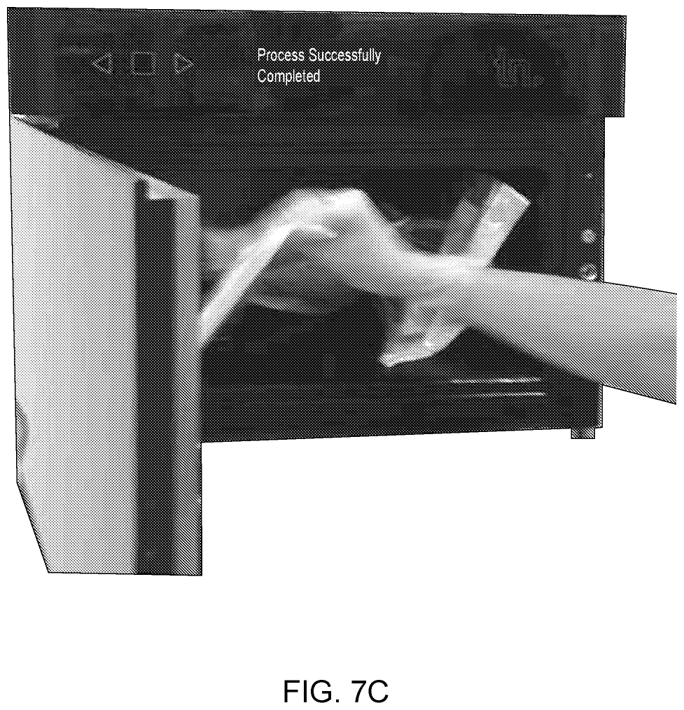

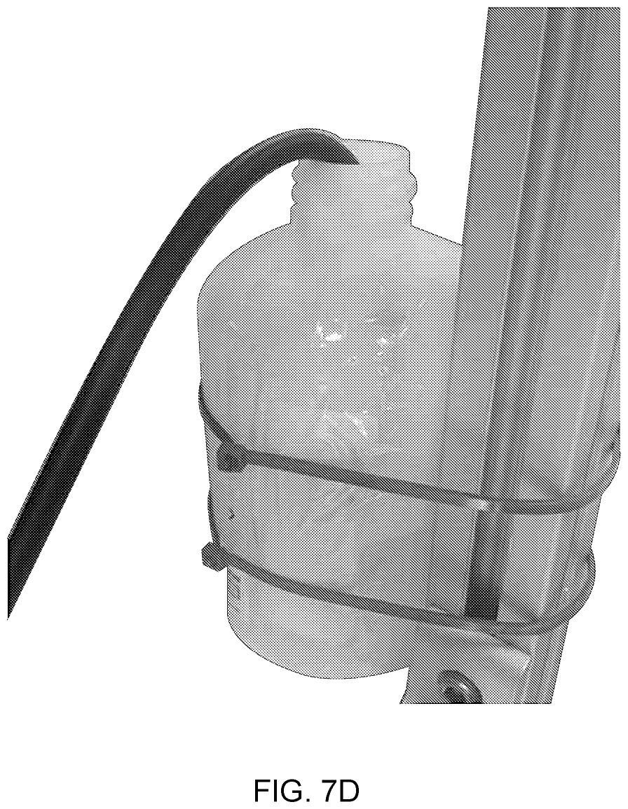

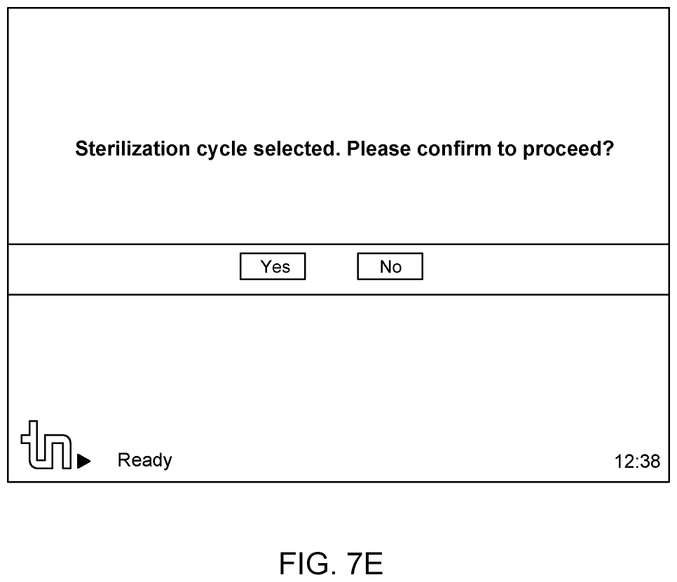

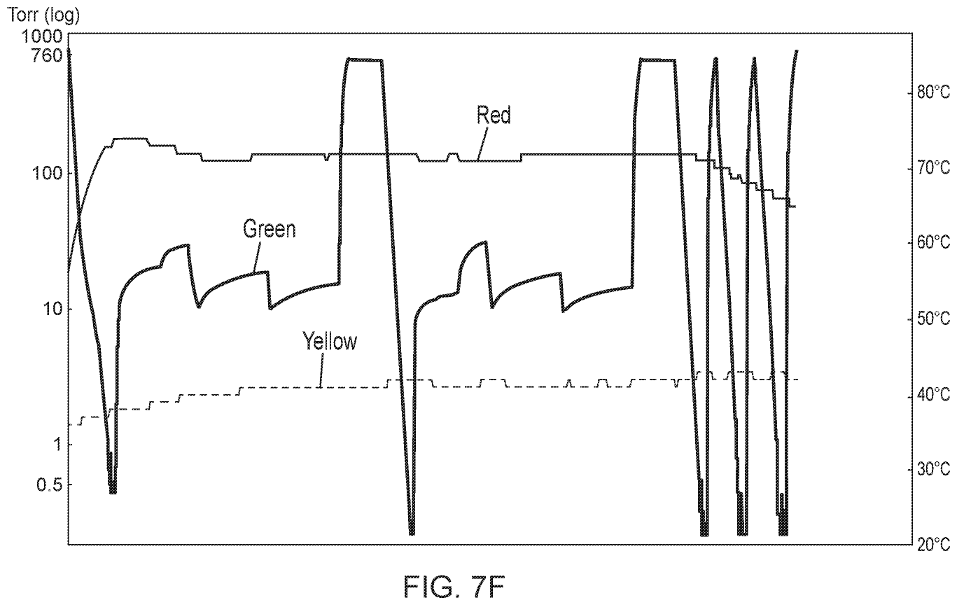

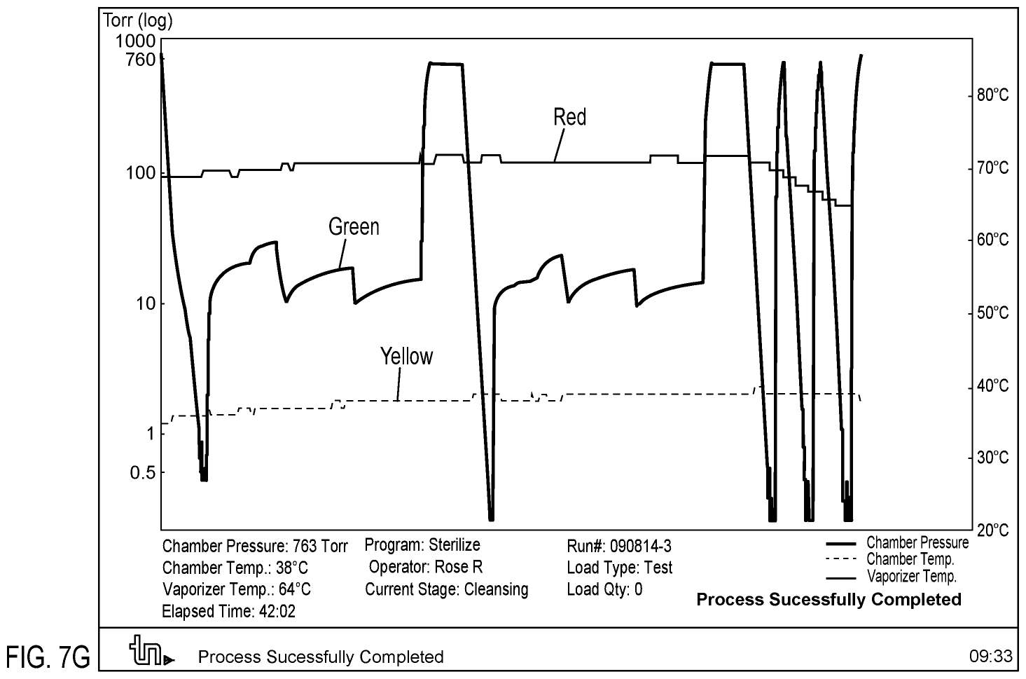

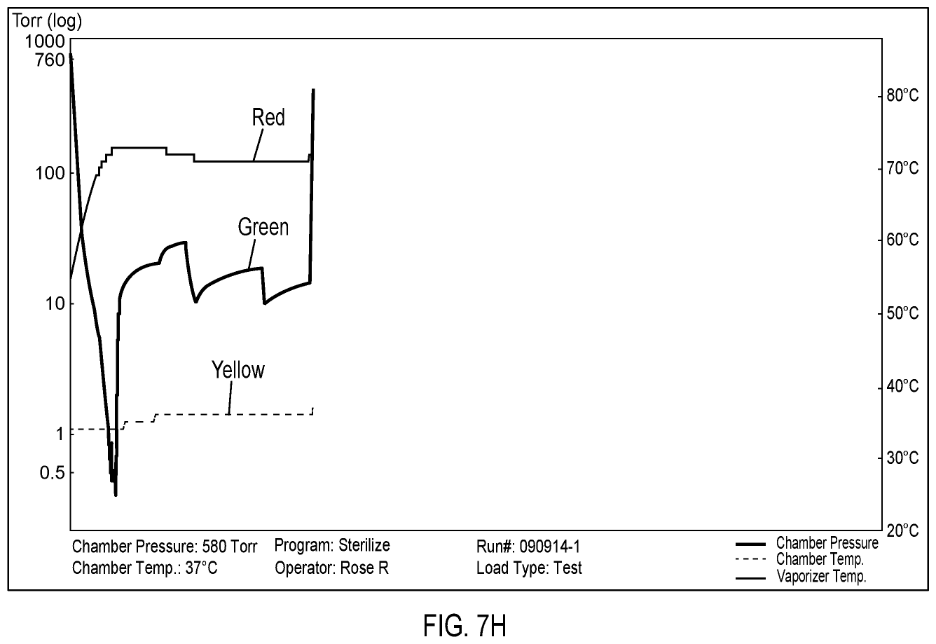

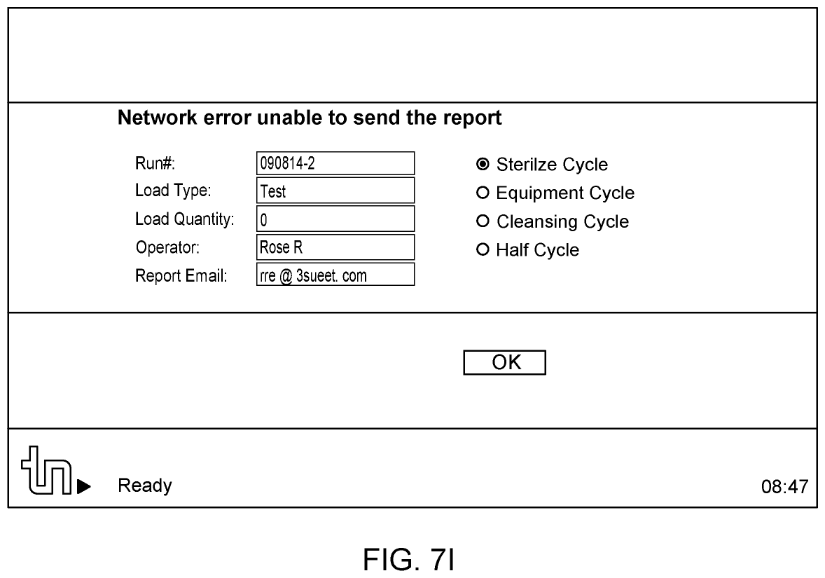

FIGS. 7A-7I show an example implementation of a system for purifying, hydrating and/or infusing organic and biological material, including an apparatus having a user interface, menu and process screens, a vacuum chamber, and a reagent container, according to some embodiments. The system of FIGS. 7A-7I has the ability to perform various functions, including: (1) purification of pathogens (including, but not limited to: mold, yeast, bacteria, fungi, and viruses), (2) removal of non-pathogens, (3) rehydration of a material, to restore or increase a moisture level thereof, and (4) infusion of one or more substances (including, but not limited to: terpenes, organic essential oils, and other liquids to enhance terpene profiles in dried cannabis flower).

The system of FIGS. 7A-7I includes stand-alone hardware that is operated in a closed environment, embedded software and accessories for using (1) reactive oxygen for purification of the material, (2) DI, IO or other types of water or solvent for rehydration of the material, and/or (3) one or more of a variety of cannabis/non-cannabis derived terpenes and organic (non-alcohol) essential oils and nutraceutical based products (e.g., for infusion). The foregoing materials (1)-(3) may collectively be referred to as “reagents.” The reagents can be further concentrated (as compared with a starting concentration thereof), vaporized, and/or injected within the vacuum chamber in a factory validated closed-loop process referred to herein as a “cycle.” The cycle process(es) can inactivate microorganisms, and rehydrate and/or infuse dried cannabis flower (or other plant or plant-derived material) while maintaining safe conditions. The cycle process can be predefined/pre-programmed and controlled using a programmable logic controller (PLC) of the system. All interactions with the PLC and control over cycles can be performed using the HMI located on the face of the machine (shown, for example, in FIG. 7C and FIG. 4). Cycle and infusion efficacy can depend on processing time, temperature, and/or pressure. Upon completion of a cycle process, remaining/residual/excess vapor can be removed from the cycle environment (e.g., the vacuum chamber) and safely decomposed to atmosphere by catalytic reactions.

System functions that are programmable and/or usable by an operator can be hosted inside the apparatus (e.g., stored within a memory that is operably coupled to a processor). The functions can be accessed by authorized representatives (e.g., in response to their entry, via the user interface, of authorized user credentials, and/or by the removal of an enclosure of the apparatus).

The apparatus includes an HMI with a color display, for operation purposes. An operator can define, initiate and/or terminate processes using the HMI panel, for example by inputting process selections. See FIG. 7A (showing an operator station/monitor) and FIG. 7B (showing an example user interface with a main menu).

The vacuum chamber shown in FIG. 7C is constructed from aluminum, for durability and ease of cleaning. An interior volume of the vacuum chamber can be accessed through the front door (shown ajar in FIG. 7C). The chamber door is mounted on hinges, and can be held shut by magnets when at atmospheric pressure and/or under vacuum during device operation.

The apparatus can include an onboard compute device having Wi-Fi connectivity capability, for example to facilitate communications therewith for one or more of: daily use and operation, uploading and/or downloading of process records, software updates, remote diagnostics, and the emailing (or other transmission) of cycle/process records, for example to a facility manager.

A complete run of the system can be referred to as a process cycle. Throughout a process cycle, the device achieves or satisfies predetermined parameters (e.g., pressure, flow rate and/or temperature setpoints). A set of cycle and safety parameters aggregated to form a process cycle are called a process cycle recipe. Different programs can be accessible via the HMI and selectable by the operator, for the processing of a desired material.

A process cycle can include one or more of the following processes, in any combination and order:
Vacuum Pump & Leak Diagnostics: The device chamber is pumped to create a vacuum. Excess humidity is removed. Several automatic diagnostics of device components are performed.
Injection: Vaporized reagent is injected into the chamber in precisely controlled quantities.
Diffusion: The load (material) is exposed to the vaporized reagent. This step can be repeated (e.g., three times) during a single full cycle.
Cleansing: The cycle chamber and the cycle load within are cleansed to remove residual reagents.

During operation the apparatus consumes a reactive oxygen solution (see, e.g., FIG. 7D). The cycle reagent can be positioned within the apparatus enclosure, affixed to an exterior of the enclosure, or exterior to the apparatus. Replacement of the cycle reagent can include the following steps (by way of example only):
Step 1: Open the right side door of the apparatus, or observe an exterior right-hand side of the apparatus.
Step 2: Depress the stainless steel reagent connection button located on the reagent connection valve.
Step 3: Remove the existing reagent.
Step 4: Ensure that the replacement reagent is within the expiration period and meets all specifications.
Step 5: Remove the container cap with drip and connection tubing, for reuse with the new container.
Step 6: Using the male side of the reagent connection tubing, firmly press into female tubing receptacle until it clicks.
Step 5: Record the date on which the reagent was opened and the initials of the operator.
Step 6: By pressing start, the device will automatically prime reagent for use.

In some embodiments, the cycle load/material and/or packaging thereof does not contain any hygroscopic materials or materials made from cellulose. alternatively or in addition, the cycle load/material is not wet, nor does it contain liquids.

In some embodiments, a cannabis flower loads is dried and packaged, being dry and free of foreign objects or non-approved packaging, prior to being processed by the system. A prepared load may allow for sufficient clearance/space for air to circulate, during the reagent diffusion process, without overcrowding the chamber within. For example, the vacuum chamber may be filled with a load/material to any degree up to, but not exceeding, 85% of capacity.

In some embodiments, the packaging is breathable and/or does not contain hygroscopic materials such as cellulose. For example, the packaging material can include a mesh, Tyvek®, or a polyethylene packaging material.

During use, in some embodiments, the vacuum chamber is loaded in such a manner that the cycle reagent can circulate freely therewithin, and readily diffuse into the packaging. For example, the load may be evenly/uniformly distributed within the vacuum chamber, and/or multiple discrete pouches of the load may be positioned within the vacuum chamber without being overly packed. Void space may be reserved within the vacuum chamber, to allow for proper vapor circulation. Once the system has been loaded, the cycle program menu can load on the HMI screen. An operator may then select a desired cycle program or recipe (e.g., from an assortment of pre-programmed recipes). Following program selection, a confirmation screen appears in the HMI, and the operator can verify the selected program. Upon selection of the program, a “start cycle” button will appear in the HMI, with which the operator can start the desired cycle.

A “Purify” process can target pathogens for vaporized disinfection. Suitable loads/materials include, for example, products packaged in Tyvek packaging or a mesh bag made from nylon, with no metal zippers or metal material.

An “Infuse Cleansing Cycle” can be performed after a completed run or after a failed run (e.g., after a power outage, vacuum failure, etc.), to ensure that the chamber and its contents are not flooded with reactive oxygen.

During operation, a cycle progress screen will appear after a brief warm-up period. Cycle process steps and other important cycle details are listed on the screen. Through the HMI interface, the operator can observe all individual steps of the cycle process and related parameters as they occur, for example in the form of real-time graphics. Real time process graphics show graphs of chamber pressure, chamber temperature, and vaporizer temperature over time, as associated with the various stages of the cycle process.

As shown in FIG. 7F, a cycle process begins with evacuation of the vacuum chamber, so the pressure line (labelled “green”) slopes downward from the top of the graph toward the bottom. Following evacuation, injection occurs, and the pressure line correspondingly rises. Injection is followed by three pulsed diffusion processes, forming 3 valleys and 2 additional hills. The chamber pressure is then increased to a process high, forming a steep rise in the graph. The curve shape for the first half cycle is repeated for the second half cycle. The vaporizer temperature line (labelled “red”) tracks chamber pressure on a considerably narrower scale. Typically, the vaporizer temperature falls during chamber evacuation, and rises again with pressurization of the chamber. The chamber temperature line (labelled “yellow”) also tracks chamber pressure, but within a considerably narrower band (i.e., almost steady).

A running cycle process can be aborted by an Abort button located at the lower right side of the screen (with optional subsequent verification by the operator via the HMI). When a cycle process has been aborted, the apparatus can immediately initiate the removal and completion stages (e.g., including a cleansing step, ensuring safe handling of the cycle load). As such, even after aborting the process cycle, the apparatus can remain operational to complete the cleansing phase of the cycle process. When the apparatus stops running the cycle process, a message can appear on the HMI screen to indicate a complete cycle process or an incomplete cycle process.

Upon successful completion of a validated cycle process, a green colored completion message can appear (see FIG. 7G), indicating that the cycle process has been successfully completed. The operator is then invited (e.g., via an HMI message) to open the cycle chamber door and unload the sterile load.

In the event of an unsuccessful (“failed”) cycle process, a red colored incomplete message can appear (see FIG. 7H) in the HMI display, indicating that the cycle was not successful and that the cycle load is not sterile.

Upon successful or failed completion of the cycle process, a cycle report can be compiled and, optionally, sent over a communications network (e.g., to a mobile or other type of remote compute device). Should the apparatus experience difficulty in connecting to the network, a network connection error can be generated in the HMI display (see FIG. 7I). However, even if the network sending of the report fails, the report can be retained/stored in the apparatus memory (or a memory that is otherwise in communication therewith) for future access.

In some embodiments, the apparatus can maintain backups of sterilizer and cycle related data, e.g., for documentation purposes. The report files can be automatically generated, and can be emailed to a preset email account. An example of a successfully completed cycle report is shown below.

Icetech Cycle Report Sep. 8, 2014 08:50

Program: Bone
Run#: 090814-3
Load Type: Test
Load Quantity: 0
Operator: Rose Regueiro
Device Run#: 104
00:00 Started Initialization >>
00:00 Injection Check 230 >>
00:00 Started Vacuum Pumping >>
00:02 Door Test in 2 sec. >>
01:12 Seal Test in 64 sec. >>
02:16 Humidity Test in 64 sec. >>
02:46 Started Injection >>
02:47 Pressure prior to 1st Injection 0 Torr >>
03:24 Pressure Rise after 1st Injection 11 Torr >>
05:23 Pressure prior to 2nd Injection 19 Torr >>
06:00 Pressure Rise after 2nd Injection 2 Torr >>
07:01 Started Diffusion >>
07:01 Pre-Separation Pressure 24 Torr >>
07:33 Separation Pump in 31 sec. >>
11:41 Separation Pump in 7 sec. >>
16:07 Completed 1st Half in 28 sec. >>
18:07 Started Pumping >>
19:27 Residue Test in 80 sec. >>
19:37 Started Injection >>
19:38 Pressure prior to 1st Injection 8 Torr >>
20:35 Pressure Rise after 1st Injection 11 Torr >>
22:35 Pressure prior to 2nd Injection 18 Torr >>
23:12 Pressure Rise after 2nd Injection 4 Torr >>
24:12 Started Diffusion >>
24:12 Pre-Separation Pressure 21 Torr >>
24:28 Separation Pump in 12 sec. >>
28:33 Separation Pump in 7 sec. >>
32:30 Completed 2nd Half in 35 sec. >>
36:00 Started Cleansing >>
36:15 1st Cleansing Pump in 75 sec. >>
37:18 1st Cleansing Vent in 27 sec. >>
38:31 2nd Cleansing Pump in 72 sec. >>
38:35 2nd Cleansing Vent in 27 sec. >>
40:49 3rd Cleansing Pump in 73 sec. >>
41:52 3rd Cleansing Vent in 37 sec. >>
42:02 Completing Cycle in 0 sec. >>
42:02 Process Successfully Completed >>

### Example Successfully Completed Cycle Report

A cycle report can include timestamps and associated critical events and parameters throughout the cycle process. When a critical event and/or parameter was successfully completed/passed, it is marked with a pass (>>) sign. When such an event or parameter fails, it is marked “FAIL” An example of a failed cycle report is shown below. Failed events can be used, for example, for the diagnosis of the failed process cycles.

Icetech Cycle Report Sep. 8, 2014 08:50

Program: Bone
Run#: 090814-3
Load Type: Test
Load Quantity: 0
Operator: Rose Regueiro
Device Run#: 104
00:00 Started Initialization >>
00:00 Injection Check 230 >>
00:00 Started Vacuum Pumping >>
00:02 Door Test in 2 sec. >>
01:12 Seal Test in 64 sec. >>
02:16 Humidity Test in 64 sec. >>
02:46 Started Injection >>
02:47 Pressure prior to 1st Injection 0 Torr >>
03:24 Pressure Rise after 1st Injection 11 Torr >>
05:23 Pressure prior to 2nd Injection 19 Torr >>
06:00 Pressure Rise after 2nd Injection 2 Torr >>
07:01 Started Diffusion >>
07:01 Pre-Separation Pressure 24 Torr >>
07:33 Separation Pump in 31 sec. >>
11:41 Separation Pump in 7 sec. >>
16:07 Completed 1st Half in 28 sec. >>
18:07 Started Pumping >>
19:27 Residue Test failed after 120 sec. >FAIL
19:28 Process Failed

### Example Failed Cycle Report

Some embodiments of the disclosure include a method, comprising: heating a vacuum chamber to a first predetermined temperature; providing an organic plant material within the vacuum chamber, the organic material having a moisture content of from about 1% to about 40%; heating a vaporizer to a second predetermined temperature, the vaporizer in fluid communication with the vacuum chamber; performing, via a vacuum pump, a first evacuation of the vacuum chamber to a first predetermined, sub-atmospheric pressure; injecting a liquid reagent into the vaporizer such that the liquid reagent transforms into a gaseous/aerosolized reagent; introducing the gaseous/aerosolized reagent into the vacuum chamber; waiting a predetermined duration so as to achieve a sterilization of the organic plant material; performing a first venting of the vacuum chamber to atmospheric pressure; performing, via the vacuum pump, a second evacuation of the vacuum chamber to a second predetermined, sub-atmospheric pressure so as to remove a reagent residue from the organic plant material; and performing a second venting of the vacuum chamber to atmospheric pressure. In some implementations, the method further comprises performing, via the vacuum pump, a third evacuation of the vacuum chamber to a second predetermined, sub-atmospheric pressure so as to remove a reagent residue; and performing a third venting of the vacuum chamber to atmospheric pressure. In some embodiments, the sterilization comprises or results in at least a 50% bioburden reduction (reduction of harmful microbes such as mold, bacteria, fungus, etc.), at least a 60% bioburden reduction, at least a 70% bioburden reduction, at least a 80% bioburden reduction, at least a 90% bioburden reduction, at least a 95% bioburden reduction, at least a 97% bioburden reduction, at least a 98% bioburden reduction, at least a 99% bioburden reduction, at least a 99.5% bioburden reduction, and/or at least a 99.9% bioburden reduction. In some embodiments, a mold count is reduced to less than 50,000 CFU, less than 25,000 CFU, less than 10,000 CFU, less than 5,000 CFU, less than 1,000 CFU, less than 500 CFU, less than 100 CFU, less than 50 CFU, and/or less than 10 CFU.

Some embodiments of the disclosure include a method, comprising: heating a vacuum chamber to a first predetermined temperature; providing an organic plant material within the vacuum chamber, the organic plant material having a moisture content of from about 0% to about 40%; heating a vaporizer to a second predetermined temperature, the vaporizer in fluid communication with the vacuum chamber; performing, via a vacuum pump, a first evacuation of the vacuum chamber to a first predetermined, sub-atmospheric pressure; injecting a liquid supplement into the vaporizer such that the liquid supplement transforms into a gaseous/aerosolized supplement; introducing the gaseous/aerosolized supplement into the vacuum chamber; and waiting a predetermined duration so as to achieve a infusion and/or saturation of the organic plant material with the supplement. In some embodiments, the supplement includes at least one of: a cannabinoid oil, a terpene, a terpinoid, a flavonoid, a cannaflavin, tetrahydrocannabinol (THC), and/or cannabidiol (CBD). In some embodiments, the organic plant material is cannabis plant material, including one or more of raw cannabis plant material, dried cannabis plant material, and/or cannabis flower.

Some embodiments of the disclosure include a method of reducing the bioburden of cannabis material and infusing said cannabis material with natural cannabis extracts to provide a sanitized organic cannabis product, the method comprising: obtaining organic cannabis material; processing the organic cannabis material such that the organic cannabis material has a moisture level between about 10% and about 16%; heating a pressure chamber to a first predetermined temperature via a first heater; inserting the organic cannabis material into the pressure chamber; heating a vaporizer via a second heater to a second predetermined temperature, the vaporizer in fluid communication with the pressure chamber; performing a first pressure change of the pressure chamber to a first predetermined pressure, the first predetermined pressure being a sub-atmospheric pressure; introducing a purifying, oxygen-based reagent into the pressure chamber via the heated vaporizer such that the purifying, oxygen-based reagent is in at least one of an aerosol, vapor, and/or gas form; processing the organic cannabis material in the pressure chamber with the purifying, oxygen-based reagent for at least one cycle having a predetermined duration, the processing reducing the bioburden of the organic cannabis material without irradiation; performing a first venting of the pressure chamber, the first venting raising the pressure of the pressure chamber to atmospheric pressure; performing at least one second pressure change of the pressure chamber to a second predetermined pressure to remove residue of the purifying, oxygen-based reagent from the organic cannabis material, the second predetermined pressure being a sub-atmospheric pressure; performing a second venting of the pressure chamber, the second venting raising the pressure of the pressure chamber to atmospheric pressure; heating the pressure chamber to a third predetermined temperature via the first heater; heating the vaporizer to a fourth predetermined temperature via the second heater; performing at least one third pressure change of the pressure chamber to a third predetermined pressure, the third predetermined pressure being a sub-atmospheric pressure; introducing a supplement into the pressure chamber via the vaporizer, the supplement being one or more natural cannabis extracts or components thereof; processing the organic cannabis material with the supplement in the pressure chamber for at least one infusion cycle having duration such that the organic cannabis material is infused with the one or more natural cannabis extracts or components thereof to produce a sanitized organic cannabis product; and outputting the sanitized organic cannabis product from the pressure chamber.

All combinations of the foregoing concepts and additional concepts illustrated (provided such concepts are not mutually inconsistent) are contemplated as being part of the disclosure. The terminology explicitly employed herein that also may appear in any disclosure incorporated by reference should be accorded a meaning most consistent with the particular concepts disclosed herein.

The drawings are primarily for illustrative purposes and are not intended to limit the scope of the disclosure. The drawings are not necessarily to scale; in some instances, various aspects of the disclosure may be shown exaggerated or enlarged in the drawings to facilitate an understanding of different features.

In order to address various issues and advance the art, the entirety of this application (including any Cover Page, Title, Headings, Background, Summary, Brief Description of the Drawings, Detailed Description, Embodiments, Numbered Embodiments, Abstract, Figures, Appendices, and otherwise) shows, by way of illustration, various embodiments in which the disclosed innovations can be practiced. The advantages and features of the application are of a representative sample of embodiments only, and are not exhaustive and/or exclusive. They are presented to assist in understanding and teach the disclosed principles.

It should be understood that the examples and embodiments are not representative of all innovations within the scope of the disclosure. As such, certain aspects of the disclosure have not been detailed herein. That alternate embodiments may not have been presented for a specific portion of the innovations or that further undescribed alternate embodiments may be available for a portion is not to be considered a disclaimer of those alternate embodiments. It will be appreciated that many of those embodiments incorporate the same principles of the innovations and others are equivalent. Thus, it is to be understood that other embodiments may be utilized and functional, logical, operational, organizational, structural and/or topological modifications may be made without departing from the scope and/or spirit of the disclosure. As such, all examples and/or embodiments are deemed to be non-limiting throughout this disclosure.

Also, no inference should be drawn regarding those embodiments discussed herein relative to those not discussed herein other than it is as such for purposes of reducing space and repetition. For instance, it is to be understood that the logical and/or topological structure of any combination of any components (a component collection), other components and/or any present feature sets as described in the figures and/or throughout are not limited to a fixed operating order and/or arrangement, but rather, any disclosed order is exemplary and all equivalents, regardless of order, are contemplated by the disclosure.

Various inventive concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, and may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently set forth in specific embodiments. Applicant reserves all rights in those innovations including the right to claim such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or examples on equivalents to the embodiments. Depending on the particular desires and/or characteristics of an implementation, various embodiments of the technology disclosed herein may be implemented in a manner that enables a great deal of flexibility and customization as described herein. Patents, patent applications, patent application publications, journal articles and protocols referenced herein are incorporated by reference in their entireties, for all purposes

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of” “Consisting essentially of,” when used in claims, shall have its ordinary meaning as used in the field of patent law.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the terms “herb”, “herbs” and “herbal” all refer to an annual, biennial, or perennial plant that does not develop persistent woody tissue but dies down at the end of a growing season. Herbal plants typically are capable of flowering and producing seeds. In some contexts, the terms refer to a plant or plant part valued for its medicinal, savory, or aromatic qualities. Examples of herbs include, but are not limited to, sage, rosemary, parsley, basil, catnip and cannabis (i.e., hemp and marijuana).

As used herein, the terms “herbal composition” or “herbal product” refer to herbs, herbal materials, herbal preparations, and finished herbal products that contain parts of plants, other plant materials, or combinations thereof as active ingredients, including for use as a medicinal, food supplement, food additive, or the like. Herbs include crude plant material, for example, leaves, flowers, fruit, seed, and stems. Herbal materials include, in addition to herbs, fresh juices, gums, fixed oils, essential oils, resins, and dry powders of herbs. Herbal preparations are the basis for finished herbal products and may include comminuted or powdered herbal materials, or extracts, tinctures, and fatty oils of herbal materials. Finished herbal products consist of herbal preparations made from one or more herbs. See, e.g., Perspectives in Clinical Research, April-June 2016, 7(2):59-61.

As used herein, “spice” or “spices” refer to an aromatic or pungent plant (e.g., an herbal or vegetable substance) used as a flavoring and/or to flavor food, e.g., cloves, pepper, or mace. A spice comprises a whole plant or a part of a plant, and/or a powder made from that whole plant or plant part.

All transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising:
heating a vacuum chamber to a first predetermined temperature;
providing an organic plant material within the vacuum chamber, the organic plant material having a moisture content of from about 1% to about 40%;
heating a vaporizer to a second predetermined temperature, the vaporizer in fluid communication with the vacuum chamber;
performing, via a vacuum pump, a first evacuation of the vacuum chamber to a first predetermined, sub-atmospheric pressure;
injecting a liquid reagent into the vaporizer such that the liquid reagent transforms into a gaseous/aerosolized reagent;
introducing the gaseous/aerosolized reagent into the vacuum chamber;
waiting a predetermined duration so as to achieve a sterilization of the organic plant material;
performing a first venting of the vacuum chamber to atmospheric pressure;
performing, via the vacuum pump, a second evacuation of the vacuum chamber to a second predetermined, sub-atmospheric pressure so as to remove a reagent residue from the organic plant material; and
performing a second venting of the vacuum chamber to atmospheric pressure.

2. The method of claim 1, further comprising:
performing, via the vacuum pump, a third evacuation of the vacuum chamber to a second predetermined, sub-atmospheric pressure so as to remove a reagent residue; and
performing a third venting of the vacuum chamber to atmospheric pressure.

3. The method of claim 1, wherein the sterilization comprises at least a 50% bioburden reduction.

4. The method of claim 1, wherein the sterilization comprises at least a 60% bioburden reduction.

5. The method of claim 1, wherein the sterilization comprises at least a 70% bioburden reduction.

6. The method of claim 1, wherein the sterilization comprises at least a 80% bioburden reduction.

7. The method of claim 1, wherein the sterilization comprises at least a 90% bioburden reduction.

8. The method of claim 1, wherein the sterilization comprises at least a 95% bioburden reduction.

9. The method of claim 1, wherein the sterilization comprises at least a 97% bioburden reduction.

10. The method of claim 1, wherein the sterilization comprises at least a 98% bioburden reduction.

11. The method of claim 1, wherein the sterilization comprises at least a 99% bioburden reduction.

12. The method of claim 1, further comprising hydrating the organic plant material while the organic plant material is disposed within the vacuum chamber, and one of prior to or subsequent to the sterilization of the organic plant material.

13. The method of claim 1, wherein the sterilization comprises at least a 99.9% bioburden reduction.

14. The method of claim 1, wherein a mold count is reduced to less than 50,000 CFU.

15. The method of claim 1, wherein a mold count is reduced to less than 25,000 CFU.

16. The method of claim 1, wherein a mold count is reduced to less than 10,000 CFU.

17. The method of claim 1, wherein a mold count is reduced to less than 5,000 CFU.

18. The method of claim 1, wherein a mold count is reduced to less than 1,000 CFU.

19. The method of claim 1, wherein a mold count is reduced to less than 500 CFU.

20. The method of claim 1, wherein a mold count is reduced to less than 100 CFU.

21. The method of claim 1, wherein a mold count is reduced to less than 50 CFU.

22. The method of claim 1, wherein a mold count is reduced to less than 10 CFU.

23. A method, comprising:
heating a vacuum chamber to a first predetermined temperature;
providing an organic plant material within the vacuum chamber, the organic plant material having a moisture content of from about 0% to about 40%;
heating a vaporizer to a second predetermined temperature, the vaporizer in fluid communication with the vacuum chamber;
performing, via a vacuum pump, a first evacuation of the vacuum chamber to a first predetermined, sub-atmospheric pressure;
injecting a liquid supplement into the vaporizer such that the liquid supplement transforms into a gaseous/aerosolized supplement;
introducing the gaseous/aerosolized supplement into the vacuum chamber; and
waiting a predetermined duration so as to achieve a infusion and/or saturation of the organic plant material with the supplement.

24. (canceled)

25. The method of claim 23, wherein the supplement includes at least one of: a cannabinoid oil, a terpenes, a terpinoid, a flavonoid, a cannaflavin, tetrahydrocannabinol (THC), and/or cannabidiol (CBD).

26. The method of claim 23, wherein the organic plant material is cannabis plant material.

27. The method of claim 26, wherein the cannabis plant material is dried cannabis plant material.

28. The method of claim 26, wherein the cannabis plant material is raw cannabis plant material.

29. The method of claim 26, wherein the cannabis plant material includes cannabis flower.

30. An apparatus, the apparatus configured to perform the method of claim 23.

31. A method of reducing the bioburden of cannabis material and infusing said cannabis material with natural cannabis extracts to provide a sanitized organic cannabis product, the method comprising:
obtaining organic cannabis material;
processing the organic cannabis material such that the organic cannabis material has a moisture level between about 10% and about 16%;
heating a pressure chamber to a first predetermined temperature via a first heater; inserting the organic cannabis material into the pressure chamber;
heating a vaporizer via a second heater to a second predetermined temperature, the vaporizer in fluid communication with the pressure chamber;
performing a first pressure change of the pressure chamber to a first predetermined pressure, the first predetermined pressure being a sub-atmospheric pressure;
introducing a purifying, oxygen-based reagent into the pressure chamber via the heated vaporizer such that the purifying, oxygen-based reagent is in at least one of an aerosol, vapor, and/or gas form;
processing the organic cannabis material in the pressure chamber with the purifying, oxygen-based reagent for at least one cycle having a predetermined duration, the processing reducing the bioburden of the organic cannabis material without irradiation; performing a first venting of the pressure chamber, the first venting raising the pressure of the pressure chamber to atmospheric pressure;
performing at least one second pressure change of the pressure chamber to a second predetermined pressure to remove residue of the purifying, oxygen-based reagent from the organic cannabis material, the second predetermined pressure being a sub-atmospheric pressure;
performing a second venting of the pressure chamber, the second venting raising the pressure of the pressure chamber to atmospheric pressure;
heating the pressure chamber to a third predetermined temperature via the first heater; heating the vaporizer to a fourth predetermined temperature via the second heater; performing at least one third pressure change of the pressure chamber to a third predetermined pressure, the third predetermined pressure being a sub-atmospheric pressure; introducing a supplement into the pressure chamber via the vaporizer, the supplement being one or more natural cannabis extracts or components thereof;
processing the organic cannabis material with the supplement in the pressure chamber for at least one infusion cycle having duration such that the organic cannabis material is infused with the one or more natural cannabis extracts or components thereof to produce a sanitized organic cannabis product; and
outputting the sanitized organic cannabis product from the pressure chamber.
